(19) United States
(12) Patent Application Publication
(10) Pub. No.: US 2009/0018643 A1
(43) Pub. Date: Jan. 15, 2009

(54) Stents

(75) Inventors: Craig Hashi, Berkeley, CA (US); Robert O'Reilly, Lafayette, CA (US); Sing-Fatt Chin, Pleasanton, CA (US); Ajit Nair, Milpitas, CA (US)

Correspondence Address: MORGAN, LEWIS & BOCKIUS LLP (SF), One Market, Spear Street Tower, Suite 2800, San Francisco, CA, 94105, US

(73) Assignee: NanoVasc, Inc., Alameda, CA (US)

(21) Appl. No.: 12/137,504

(22) Filed: Jun. 11, 2008

Related U.S. Application Data
(60) Provisional application No. 60/943,305, filed on Jun. 11, 2007.

(51) Int. Cl. A61F 2/06

(52) U.S. Cl. 623/1.15; 623/1.44

(57) ABSTRACT

The invention provides a composition comprising a stent and a polymer scaffold.

### CROSS-REFERENCE TO RELATED APPLICATIONS

The present application claims priority to U.S. Provisional Application 60/943,305, filed on Jun. 11, 2008, the entire disclosures of which are incorporated herein by reference in their entirety for all purposes.

### BACKGROUND OF THE INVENTION

The use of stent medical devices, or other types of endoluminal mechanical support devices, to keep a duct, vessel or other body lumen open in the human body has developed into a primary therapy for lumen stenosis or obstruction. The use of stents in various surgical procedures has quickly become accepted as experience with stent devices accumulates, and the number of surgical procedures employing them increases as their advantages become more widely recognized. For example, it is known to use stents in body lumens in order to maintain open passageways such as the prostatic urethra, the esophagus, the biliary tract, intestines, and various coronary arteries and veins, as well as more remote cardiovascular vessels such as the femoral artery, etc. There are two types of stents that are presently utilized: permanent stents and temporary stents. A permanent stent is designed to be maintained in a body lumen for an indeterminate amount of time. Temporary stents are designed to be maintained in a body lumen for a limited period of time in order to maintain the patency of the body lumen, for example, after trauma to a lumen caused by a surgical procedure or an injury. Permanent stents are typically designed to provide long-term support for damaged or traumatized wall tissues of the lumen. There are numerous conventional applications for permanent stents including cardiovascular, urological, gastrointestinal, and gynecological applications.

Permanent and temporary stents are made from various conventional, biocompatible metals. However, there are several disadvantages that may be associated with the use of metal stents. For example, it is known that the metal stents may become encrusted, encapsulated, epithelialized or ingrown with body tissue. Moreover, the stents are known to migrate on occasion from their initial insertion location. Such stents are known to cause irritation to the surrounding tissues in a lumen. Also, since metals are typically much harder and stiffer than the surrounding tissues in a lumen, this may result in an anatomical or physiological mismatch, thereby damaging tissue or eliciting unwanted biologic responses. Although permanent metal stents are designed to be implanted for an indefinite period of time, it is sometimes necessary to remove permanent metal stents. For example, if there is a biological response requiring surgical intervention, often the stent must be removed through a secondary procedure. If the metal stent is a temporary stent, it will also have to be removed after a clinically appropriate period of time. Regardless of whether the metal stent is categorized as permanent or temporary, if the stent has been encapsulated, epithelialized, etc., the surgical removal of the stent will resultingly cause undesirable pain and discomfort to the patient and possibly additional trauma to the lumen tissue. In addition to the pain and discomfort, the patient must be subjected to an additional time consuming and complicated surgical procedure with the attendant risks of surgery, in order to remove the metal stent.

Bioabsorbable and biodegradable materials are used for manufacturing stents. The conventional bioabsorbable or bioresorbable materials from which such stents are made are selected to absorb or degrade over time, thereby eliminating the need for subsequent surgical procedures to remove the stent from the body lumen. In addition to the advantages attendant with not having to surgically remove such stents, it is known that bioabsorbable and biodegradable materials tend to have excellent biocompatibility characteristics, especially in comparison to most conventionally used biocompatible metals. Another advantage of stents made from bioabsorbable and biodegradable materials is that the mechanical properties can be designed to substantially eliminate or reduce the stiffness and hardness that is often associated with metal stents, which can contribute to the propensity of a stent to damage a vessel or lumen.

However, there are disadvantages known to be associated with the use of bioabsorbable or biodegradable stents. The disadvantages arise from the limitation of the material from which the stent is made. One of the problems associated with the current stents is that the materials break down too quickly. This improper breakdown or degradation of a stent into large, rigid fragments in the interior of a lumen, such as the urethra, may cause obstruction to normal flow, such as voiding, thereby interfering with the primary purpose of the stent in providing lumen patency. Alternatively, they take a long time to breakdown and stay in the target lumen for a considerable period of time after their therapeutic use has been accomplished.

Accordingly, there is a need in this art for novel, temporary stents made from biodegradable polymers, wherein the stents remain functional in a body lumen for the duration of a prescribed, clinically appropriate period of time to accomplish the predetermined therapeutic purpose, and, then degrade without breaking down into large, rigid fragments, which may cause irritation, obstruction, pain or discomfort to the patient.

In a preferred embodiment of the present invention, the temporary stent readily passes out of the body as very soft particles or soft fibrous element or elements, and irritation, obstruction, pain or discomfort to the patient is either eliminated, or if present, is minimal.

### SUMMARY OF THE INVENTION

Cardiovascular diseases are a leading cause of death in the United States. Stenting post-angioplasty procedures have been widely used to support diseased vessels. Metal and polymer stents have been developed as have stents with drug-release capability. However, there is still a significant percentage of recurrence of vascular diseases with current stenting procedure. Novel stent formats are needed to minimize or eliminate restenosis.

In various embodiments, the invention provides a stent of essentially tubular cross-section bounded by a first longitudinal terminus and a second longitudinal terminus. The stent comprises a stent scaffold having an intraluminal surface and an extraluminal surface; and a first nanodiameter fibrous polymer layer, comprising a plurality of nanodiameter polymer fibers, in intimate contact with a member selected from said intraluminal surface, said extraluminal surface and combinations thereof. Also provided is a stent scaffold formed from polymer fibers.

In an exemplary embodiment, the invention provides methods of making the stents and stent scaffolds of the invention.

Yet another aspect of the present invention is a method of using the stents of the present invention in a surgical procedure to maintain the patency of a body lumen. A stent of the present invention is provided. The stent is an elongate, hollow member and in a preferred embodiment has a helical structure having a plurality of coils. The member has a longitudinal axis. In various embodiments, the coils have a pitch. The structure is made from a filament or a fiber having an inner core. The inner core has an exterior surface. Optionally, the inner core is hollow. The filament or fiber also has an outer layer covering substantially all of the exterior surface of the inner core. The filament or fiber has a cross-section. The rates of degradation of the inner core and outer layer are selected to effectively provide in a preferred embodiment such that the rate of degradation of the inner core is higher than the degradation rate of the outer layer to effectively provide that the inner core degrades in vivo, and loses it's mechanical integrity and is substantially removed from the lumen prior to elimination of the degradation of the outer layer. The inner core typically degrades by hydrolysis and breaks down at a faster rate than the outer layer with exposure to body fluids; the outer layer degrades or erodes into a soft, fibrous morphology. The stent is inserted into the body lumen of a patient, thereby providing for the patency of the lumen for a specific range of times. The stent is maintained in the lumen for a sufficient period of time to effectively maintain the lumen open and to effectively let the inner core degrade such that the softened outer core may be passed through the lumen.

In other exemplary embodiments, the invention provides a stent as described herein and further comprising a biomolecule, wherein the biomolecule is non-covalently associated or covalently attached, either directly or through a linker, to a first fibrous polymer scaffold or layer of the stent. In an exemplary embodiment, the biomolecule is an antiplatelet agent. In an exemplary embodiment, the fiber or fibers of the first fibrous polymer scaffold are aligned.

Other aspects, embodiments and objects of the present invention are apparent from the detailed description which follows.

### BRIEF DESCRIPTION OF THE DRAWINGS

FIG. 1 refers to a schematic of the electrospinning apparatus with mandrel 56A.

FIG. 2 refers to a perspective view of the electrospinning apparatus with mandrel 56A.

FIG. 2A refers to a portion of the electrospinning apparatus with mandrel 56A forming polymer scaffold 90.

FIG. 2B refers to a portion of the electrospinning apparatus with mandrel 56A forming polymer scaffold 90.

FIG. 3 illustrates various mandrel designs used for fabricating fibrous polymer scaffolds. (A) mandrel 56 in which the entire surface is conducting; (B) mandrel 56A with a first conducting region 57A, a second conducting region 57B, a non-conducting region 55, a first interface 55A, and a second interface 55B; (C) cross section of mandrel 56A in which non-conducting region 55 interconnects the two conducting mandrel regions; (D) cross section of mandrel 56A in which non-conducting region 55A is a sleeve which covers a portion of the surface of the conducting portion 57; (E) mandrel 56B with a first conducting region 57A, a second conducting region 57B, a first conducting region face 57C, a second conducting region face 57D. The conduction regions are separated by an air gap 58.

FIG. 4A is an illustration of a conduit polymer scaffold composed of longitudinally aligned micro/nanofibers. FIG. 4B is an illustration of a rod polymer scaffold composed of longitudinally aligned micro/nanofibers. Note: fiber dimensions not drawn to scale.

FIG. 5A is an illustration showing a cross section of the conduit in FIG. 4A. FIG. 5B is an illustration showing a cross section of the rod in FIG. 4B.

FIG. 6 refers to a schematic of the electrospinning apparatus with mandrel 56B.

FIG. 7 refers to a perspective view of the electrospinning apparatus with mandrel 56B.

FIG. 7A refers to a portion of the electrospinning apparatus with mandrel 56B forming polymer scaffold 92.

FIG. 8 is an illustration of a longitudinally aligned polymer scaffold sheet 96.

FIG. 9 is a schematic diagram showing the rolling process for creating a fibrous polymer conduit scaffold with a seam from an aligned polymer scaffold sheet. Here a longitudinally aligned polymer scaffold sheet 96 is rolled around a rod 97 and later sutured or adhered.

FIG. 10 is an illustration of a ‘criss-cross’ sheet 102 which comprises aligned sheets 96 and 100.

FIG. 11 refers to a schematic of a multiple spinneret electrospinning apparatus 110 with mandrel 56B. The polymer solutions 38, 38A and 38B contain the polymer dissolved in a solvent, are contained within syringe assemblies 36, 36A and 36B, respectively. The syringe assemblies are part of a syringe pump assembly 32 in which a computer 34 controls the rate at which the polymer solution exits the syringe by controlling pressure or flow rate. Optionally, different flow rates can be provided and controlled to selected spinnerets. The flow rate will change depending on the desired physical characteristics of the polymer scaffold, i.e., membrane thickness, fiber diameter, pore size, membrane density, etc.

The syringe pump assembly 32 feeds the polymer solutions to spinnerets 42, 42A and 42B that sit on a platform 44. The spinnerets have a tip geometry which allows for jet formation and transportation, without interference. A charge in the range of about 10 to about 30 kV is applied to the spinnerets by a high voltage power supply 48 through wire 41A.

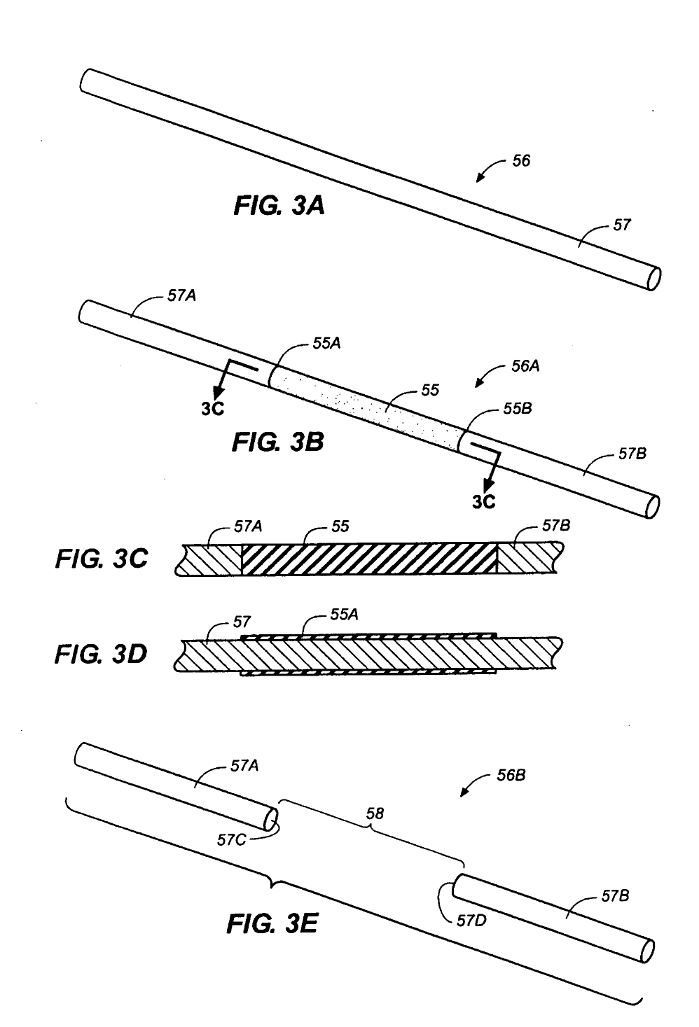

A mandrel 56B (which, as mentioned in FIG. 3B, includes 57A, 57B and 58) is positioned below the spinnerets 42, 42A and 42B such that an electric field is created between the charged spinneret and the mandrel 56A. The electric field causes a jet of the polymer solution to be ejected from the spinnerets and spray towards the mandrel 56B, forming micron or nanometer diameter filaments or fibers 46, 46A and 46B. The drill chucks are grounded using ground wires 41B and 41C.

The mandrel 56B is attached to a first drill chuck 54 (attached to a non-conducting bearing 60) and a second drill chuck 54A (attached to a non-conducting bearing 60A) which is connected to a motor 52. The motor 52 is linked to a speed control 50A which controls the rate at which the motor spins the mandrel 56B. Optionally, different spin rates can be provided. The spin rate will change depending on the desired physical characteristics of the polymer scaffold, i.e., membrane thickness, fiber diameter, pore size, membrane density, etc.

FIG. 12 is a schematic drawing of a device of the invention. A nanofibrous layer 75 is integrated on the surface of the stent.

FIG. 13 is a schematic drawing of a device of the invention showing the stent scaffold 70 in the device.

FIG. 14 is a drawing showing the deposition of a nanodiameter fiber layer 75 on a stent scaffold 70 in contact with a mandrel 56B of an electrospinning device.

FIG. 15 is a drawing showing an electrospinning device set up for deposition of a nanodiameter fiber layer on a stent scaffold 70 in which the stent scaffold replaces the mandrel of an electrospinning device

FIG. 16 is a drawing showing the deposition of a nanodiameter fiber layer 75 on a stent scaffold 70 in which the stent scaffold replaces the mandrel of an electrospinning device.

FIG. 17 is a drawing showing an electrospinning device set up to accept a nanodiameter polymer layer on the mandrel of the electrospinning device.

FIG. 18 is a drawing showing a mandrel of an electrospinning device on which a layer of a nanodiamter polymer material 75 is deposited.

FIG. 19 is a drawing showing a layer of a nanodiameter polymer material 75 deposited on a mandrel of an electrospinning device in contact with a stent scaffold 70.

FIG. 20 is a drawing showing a layer of a nanodiameter polymer material deposited 75 on a mandrel of an electrospinning device in contact with a stent scaffold 70 onto which a second layer of a nanodiameter polymer material 80 is deposited.

FIG. 21 is a drawing showing a deposition device 90 for a nanodiameter polymer layer inserted into the intraluminal space of a stent scaffold 70 prior to deposition of the polymer layer on this surface.

FIG. 22 is an close up view of a deposition device 90 for a nanodiameter polymer layer showing ports 100 through which fibers of polymer material are delivered.

### DETAILED DESCRIPTION OF THE PREFERRED EMBODIMENTS

### Definitions and Abbreviations

The abbreviations used herein generally have their conventional meaning within the chemical and biological arts.

As used herein the singular forms “a”, “and”, and “the” include plural referents unless the context clearly dictates otherwise.

Stents, grafts, stent-grafts, vena cava filters, expandable frameworks, and similar implantable medical devices, collectively referred to hereinafter as stents, are radially expandable endoprostheses which are typically intraluminal (e.g., intravascular) implants capable of being implanted transluminally and enlarged radially after being introduced percutaneously. Stent scaffolds (and the stents formed therefrom) may be self-expanding, expanded by an internal radial force, such as when mounted on a balloon, or a combination of self-expanding and balloon expandable (hybrid expandable). Stent scaffolds may be created by methods including cutting or etching a design from a tubular stock, from a flat sheet which is cut or etched and which is subsequently rolled or from one or more interwoven wires or braids. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system.

As used herein, and unless otherwise indicated, a composition that is “essentially free” of a component means that the composition contains less than about 20% by weight, such as less than about 10% by weight, less than about 5% by weight, or less than about 3% by weight of that component.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot) or another moiety.

“Antibody,” as used herein, generally refers to a polypeptide comprising a framework region from an immunoglobulin or fragments or immunoconjugates thereof that specifically binds and recognizes an antigen. The recognized immunoglobulins include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. As used herein, the term “copolymer” describes a polymer which contains more than one type of subunit. The term encompasses polymer which include two, three, four, five, or six types of subunits.

The term “isolated” refers to a material that is substantially or essentially free from components, which are used to produce the material. The lower end of the range of purity for the compositions is about 60%, about 70% or about 80% and the upper end of the range of purity is about 70%, about 80%, about 90% or more than about 90%.

“Hydrogel” refers to a water-insoluble and water-swellable cross-linked polymer that is capable of absorbing at least 3 times, preferably at least 10 times, its own weight of a liquid. “Hydrogel” and “thermo-responsive polymer” are used interchangeably herein. The term “attached,” as used herein encompasses interaction including, but not limited to, covalent bonding, ionic bonding, chemisorption, physisorption and combinations thereof.

The term “biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

“Small molecule,” refers to species that are less than 1 kD in molecular weight, preferably, less than 600 D.

Where substituent groups are specified by their conventional chemical formulae, written from left to right, they equally encompass the chemically identical substituents, which would result from writing the structure from right to left, e.g., —CH₂O— is intended to preferably also recite —OCH₂—.

By “effective” amount of a drug, formulation, or permeant is meant a sufficient amount of a active agent to provide the desired local or systemic effect. A “therapeutically effective” amount refers to the amount of drug needed to effect the desired therapeutic result.

The term “pharmaceutically acceptable salts” is meant to include salts of the compounds of the invention which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the present invention provides compounds which are in a prodrug form. Prodrugs of the compounds or complexes described herein readily undergo chemical changes under physiological conditions to provide the compounds of the present invention. Additionally, prodrugs can be converted to the compounds of the present invention by chemical or biochemical methods in an ex vivo environment.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “pharmaceutically acceptable carrier” or “pharmaceutically acceptable vehicle” refers to any formulation or carrier medium that provides the appropriate delivery of an effective amount of a active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

“Pharmaceutically acceptable topical carrier” and equivalent terms refer to pharmaceutically acceptable carriers, as described herein above, suitable for topical application. An inactive liquid or cream vehicle capable of suspending or dissolving the active agent(s), and having the properties of being nontoxic and non-inflammatory when applied to the skin, nail, hair, claw or hoof is an example of a pharmaceutically-acceptable topical carrier. This term is specifically intended to encompass carrier materials approved for use in topical cosmetics as well.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to the subject.

The term “excipients” is conventionally known to mean carriers, diluents and/or vehicles used in formulating drug compositions effective for the desired use.

The term “autologous cells”, as used herein, refers to cells which are a subject's own cells, or clones thereof.

The term “allogeneic cells”, as used herein, refers to cells which are not a first subject's own cells, or clones thereof, but are cells, or clones thereof, derived from a second subject and this second subject is of the same species as the first subject.

The term “heterologous cells”, as used herein, refers to cells which are not from a first subject's own cells, or clones thereof, but are cells, or clones thereof, derived from a second subject and this second subject is not the same species as the first subject.

The term “stem cells”, as used herein, refers to cells capable of differentiation into other cell types, including those having a particular, specialized function (i.e., terminally differentiated cells, such as erythrocytes, macrophages, etc.). Stem cells can be defined according to their source (adult/somatic stem cells, embryonic stem cells), or according to their potency (totipotent, pluripotent, multipotent and unipotent).

The term “unipotent”, as used herein, refers to cells can produce only one cell type, but have the property of self-renewal which distinguishes them from non-stem cells.

The term, “multipotent”, or “progenitor”, as used herein, refers to cells which can give rise to any one of several different terminally differentiated cell types. These different cell types are usually closely related (e.g. blood cells such as red blood cells, white blood cells and platelets). For example, mesenchymal stem cells (also known as marrow stromal cells) are multipotent cells, and are capable of forming osteoblasts, chondrocytes, myocytes, adipocytes, neuronal cells, and β-pancreatic islets cells. Another example is skeletal myoblasts, which preferentially give rise to skeletal muscle cells by a differentiation process involving fusion of individual cells into multinucleated myotubes.

The term “pluripotent”, as used herein, refers to cells that give rise to some or many, but not all, of the cell types of an organism. Pluripotent stem cells are able to differentiate into any cell type in the body of a mature organism, although without reprogramming they are unable to de-differentiate into the cells from which they were derived. As will be appreciated, “multipotent”/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells.

The term “totipotent”, as used herein, refers to fertilized oocytes, as well as cells produced by the first few divisions of the fertilized egg cell (e.g., embryos at the two and four cell stages of development). Totipotent cells have the ability to differentiate into any type of cell of the particular species. For example, a single totipotent stem cell could give rise to a complete animal, as well as to any of the myriad of cell types found in the particular species (e.g., humans). In this specification, pluripotent and totipotent cells, as well as cells with the potential for differentiation into a complete organ or tissue, are referred as “primordial” stem cells.

The term “dedifferentiation”, as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “re-programming” the cell to become more primitive. An example of dedifferentiation is the conversion of a myogenic progenitor cell, such as early primary myoblast, to a muscle stem cell or satellite cell.

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “growth environment” is an environment in which stem cells will proliferate in vitro. Features of the environment include the medium in which the cells are cultured, and a supporting structure (such as a substrate on a solid surface) if present.

“Growth factor” refers to a substance that is effective to promote the growth of cells and which, unless added to the culture medium as a supplement, is not otherwise a component of the basal medium. Put another way, a growth factor is a molecule that is not secreted by cells being cultured (including any feeder cells, if present) or, if secreted by cells in the culture medium, is not secreted in an amount sufficient to achieve the result obtained by adding the growth factor exogenously. Growth factors include, but are not limited to, basic fibroblast growth factor (bFGF), acidic fibroblast growth factor (aFGF), epidermal growth factor (EGF), insulin-like growth factor-I (IGF-I), insulin-like growth factor-II (IGF-II), platelet-derived growth factor-AB (PDGF), vascular endothelial cell growth factor (VEGF), activin-A, bone morphogenic proteins (BMPs), insulin, cytokines, chemokines, morphogens, neutralizing antibodies, other proteins, and small molecules.

The term “differentiation factor”, as used herein, refers to a molecule that induces a stem cell or progenitor cell to commit to a particular specialized cell type.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. Components of an extracellular matrix can include laminin, collagen and fibronectin.

The term “regenerative capacity”, as used herein, refers to the conversion of a stem cell into a dividing progenitor cell and a differentiated tissue-specific cell.

The term, “self renewal”, as used herein, refers to proliferation without lineage specification.

The term, “aligned”, as used herein, refers to the orientation of fibers in a fibrous polymer scaffold wherein at least 50% of the fibers are oriented in a general direction and their orientation forms an average axis of alignment. The orientation of any given fiber can deviate from the average axis of alignment and the deviation can be expressed as the angle formed between the alignment axis and orientation of the fiber. A deviation angle of 0° exhibits perfect alignment and 900 (or −90°) exhibits orthogonal alignment of the fiber with respect to the average axis of alignment. In an exemplary embodiment, the standard deviation of the fibers from the average axis of alignment can be an angle selected from between 0° and 1°, between 0° and 3°, between 0° and 5°, between 0° and 10°, between 0° and 15°, between 0° and 20°, or between 0° and 30°.

The term ‘rod’, as used herein, refers to a fibrous polymer scaffold which is essentially in the shape of a filled cylinder. Spaces and channels can be present between the individual fibers which compose the rod.

The term ‘conduit’, as used herein, refers to an object that is essentially cylindrical in shape. The conduit has an inner wall and an outer wall, an interior diameter, an exterior diameter, and an interior space which is defined by the inner diameter of the conduit as well as its length. Spaces and channels can be present between the individual fibers which compose the conduit.

The term ‘filled conduit’, as used herein, refers to a conduit in which a portion of the interior space is composed of filler material. This filler material can be a fibrous polymer scaffold. Spaces and channels can be present between the individual fibers which compose the filled conduit.

The term ‘seam’ or ‘seamed’, as used herein, refers to a junction formed by fitting, joining, or lapping together two sections. These two sections can be held together by mechanical means, such as sutures, or by chemical means, such as annealing or adhesives. For example, a seam is formed by joining one region of a sheet to another region.

The term ‘seamless’, as used herein, refers to an absence of a seam.

The term “cell” can refer to either a singular (“cell”) or plural (“cells”) situation.

The term “extracellular matrix component”, as used herein, is a member selected from laminin, collagen, fibronectin and elastin.

The term “stent”, as used herein, is a tube which can be made of, among other things, metal and organic polymers. When the stent is made of an organic polymer, the polymer is not a nanofibrous or microfibrous polymer scaffold as described herein. In other words, if the stent is made from a fibrous polymer scaffold, the average diameter of the fibers will be between 100 microns and about 50 centimeters. In some instances, the entire stent is capable of expanding from a first diameter to a second diameter, wherein the second diameter is greater than the first diameter.

The term “hirudin”, as used herein, refers to the 65 amino acid wild-type peptide or analogs thereof. The 65 amino acid wild-type peptide has a sequence described in Folkers et al., Biochemistry, 28(6): 2601-2617 (1989). Analogs of hirudin include peptides with one or more mutations, fewer amino acids, more amino acids, chemical modifications to one or more amino acid residues, and combinations thereof. Examples of hirudin include wild-type hirudin, bivalirudin, lepirudin, desirudin, non-sulfated Tyr-63 hirudin, hirudin with the N-terminus modified (ie acetylated), hirudin with the C-terminus modified (ie acetylated), a hirudin fragment with the N-terminal domain deleted (approximately residues 1-53), a hirudin fragment with the C-terminal domain deleted (approximately residues 54-65), [Tyr(SO₃H)-63]-hirudin fragment 54-65, [Tyr(SO₃H)-63]-hirudin fragment 55-65, acetyl [Tyr(SO₃H)-63]-hirudin fragment 54-65, acetyl[Tyr(SO₃H)-63]-hirudin fragment 55-65. Hirudin for use in this invention can be produced from a variety of sources. In some instances, hirudin is isolated from leeches. In others, hirudin is recombinantly produced from bacteria, yeast or fungi. In still others, hirudin is chemically synthesized. Recombinant and chemical syntheses tend to produce homogenous products, while hirudin isolated from leeches can include more than one hirudin analog. Hirudin is commercially available from companies such as Sigma-Aldrich (St. Louis, Mo.).

The symbol [symbol] whether utilized as a bond or displayed perpendicular to a bond, indicates the point at which the displayed moiety is attached to the remainder of the molecule, for example, a polymer.

### The Stents

In an exemplary embodiment, the invention provides a stent formed from a combination of a stent scaffold and a plurality of nanodiameter polymer fibers contacting at least one surface of the scaffold. In an exemplary embodiment, the stent is biodegradable, or at least one plurality of nanodiameter polymer fibers is biodegradable; when the stent includes two or more pluralities of nanodiameter polymer fibers, more than one are optionally biodegradable. In another exemplary embodiment, the entire stent (both the scaffold and the plurality of nanodiamter polymer fibers) are made from nanodiameter polymer fibers. In an exemplary embodiment one or both of the scaffold and the plurality of nanodiameter polymer fibers is biodegradable. The plurality of nanodiameter polymer fibers can be aligned in the longitudinal direction, or the circumferential direction.

In various embodiments, the stent provides several advantages including: (1) The plurality of nanodiameter polymer fibers mimics the native matrix fibrils for cell adhesion and migration; (2) The plurality of nanodiameter polymer fibers allows vessel remodeling and restoration; (3) The aligned plurality of nanodiameter polymer fibers limits over-growth of smooth muscle cells; (4) The aligned plurality of nanodiameter polymer fibers promotes endothelial migration and wound healing; (5) The aligned plurality of nanodiameter polymer fibers increases the surface/volume ratio and drastically increases the efficiency and capacity of drug delivery, (6) The stent including a plurality of nanodiameter polymer fibers can be used as a platform to deliver stem cells.

In an exemplary embodiment, the stent comprises a stent and at least layer of a plurality of nanodiameter polymer fibers covering a member selected from the inner surface of the stent scaffold, the outer surface of the stent scaffold and combinations thereof. In an exemplary embodiment, the stent comprises a stent scaffold and at least two layers comprising a plurality of nanodiameter polymer fibers, wherein one of the polymer layers (aligned or random) covers the inner surface of the stent scaffold and the second polymer scaffold (aligned or random) covers the outer surface of the stent scaffold as a sandwiched structure. In various exemplary embodiments, the composition comprises a stent having a stent scaffold, wherein more than one plurality of nanodiameter polymer fibers (aligned or random) covers either the inside surface or the outside surface of the stent scaffold, or both. In an exemplary embodiment, the stent includes at least one plurality of nanodiameter polymer fibers, wherein the stent scaffold (aligned or random) is etched or applied directly to one surface (either inner or outer) of the plurality of nanodiameter polymer fibers. In an exemplary embodiment, the stent comprises a stent scaffold, wherein the scaffold (aligned or random) is etched or applied directly to both the inner and outer surface of the plurality of nanodiameter polymer fibers.

### Stent Scaffold

Exemplary stent scaffolds (and the stents formed therefrom) of use in the present invention are generally cylindrically-shaped devices which function to hold open and sometimes expand a segment of a blood vessel or other lumen such as a coronary artery.

A variety of devices are known in the art for use as stents and have included balloon expandable stents having a variety of patterns; coiled wires in a variety of patterns that are expanded after being placed intraluminally on a balloon catheter; helically wound coiled springs manufactured from an expandable heat sensitive metal; and self expanding stents inserted in a compressed state and shaped in a zigzag pattern. In various embodiments, the present invention includes a stent scaffold according to any of these formats.

An exemplary stent of the invention possesses a unique set of properties; it can travel through small and tortuous body lumens to the treatment site, as well as be expanded to no more than its working diameter to provide consummate lumen expansion and radial support subsequent to implantation. Ideally, the stent and stent scaffold are formed from a material that exhibits a very high modulus of elasticity, a very low yield point, a high tensile strength, a variable work hardening rate, and good fatigue resistance, and that provides flexibility to the stent for navigating the tortuous vascular anatomy. Further, a radially-expandable stent must undergo significant plastic deformation when being expanded into its deployed state, which requires a stent material to have good elongation or ductility. Finally, in various embodiments, exemplary stent scaffolds are made of a material having a high degree of radiopacity, good corrosion resistance and biocompatibility to vascular tissue, blood and other bodily fluids.

A variety of materials can be used to form the stent scaffold. The scaffold can be made from synthetic and/or natural sources. In another exemplary embodiment, the scaffold is made of metal. In another exemplary embodiment, the metal is a member selected from cobalt and steel. In another exemplary embodiment, the metal is stainless steel. In various exemplary embodiments, the stent scaffold is constructed of a metal alloy, e.g., stainless steel, nickel-titanium (NiTi or nitinol), cobalt-chromium (MP35N), platinum, and other suitable metals.

In various embodiments, stent scaffolds of use in the present invention include a plurality of adjacent cylindrical rings which are generally expandable in the radial direction and arranged in alignment along a longitudinal stent axis. In an exemplary embodiment at least one link extends between adjacent cylindrical rings and connects them to one another. The rings and links may each be formed with a variety of undulations containing a plurality of alternating peaks and valleys. This configuration helps to ensure minimal longitudinal contraction during radial expansion of the stent in the body lumen. The undulations of the rings and links contain varying degrees of curvature in regions of the peaks and valleys and are adapted so that the radial expansion of the cylindrical rings are generally uniform around their circumferences during expansion of the stents from their contracted conditions to their expanded conditions.

The resulting stent structures are a series of radially expandable cylindrical rings which are spaced longitudinally close enough so that small dissections in the wall of a body lumen may be pressed back into position against the luminal wall, but not so close as to compromise the longitudinal flexibility of the stent both when being negotiated through the body lumens in their unexpanded state and when expanded into position. Upon expansion, each of the individual cylindrical rings may rotate slightly relative to their adjacent cylindrical rings without significant deformation, cumulatively providing stents which are flexible along their length and about their longitudinal axis, but which are still very stable in the radial direction in order to resist collapse after expansion.

In other exemplary embodiments, the stent scaffold has a coiled or serpentine architecture. In one embodiment, the invention is directed to a stent comprising a serpentine band disposed about the longitudinal axis. The serpentine band has a plurality of proximal turns and distal turns. Each strut extends between one proximal turn and one distal turn. The distal turns are arranged in a pattern of increasing and decreasing extent in a proximal direction. The pattern includes at least one set of three or more consecutive interconnected peaks and troughs of decreasing proximal extent followed by at least one set of three or more consecutive interconnected peaks and troughs of increasing proximal extent.

In exemplary embodiments, the stent scaffold is made from a fibrous polymer. In various embodiments, the average diameter of the fibers will be at least 100 microns. When the stent scaffold is made of a polymer, the polymers have an average diameter which is a member selected from between 100 microns and 50 centimeters, or between 100 microns and 5 millimeters, or between 0.3 millimeters and 1 millimeter, or between 0.5 millimeters and 4 millimeters, or between 0.3 millimeters and 3 millimeters. In an exemplary embodiment the stent is mechanically durable. In another exemplary embodiment, the entire stent is capable of expanding from a first diameter to a second diameter, wherein the second diameter is greater than the first diameter.

In various exemplary embodiments, stent scaffold (e.g., polymer stent scaffold) has a length which is a member selected from about 0.01 cm to about 20 cm, about 0.05 cm to about 5 cm, about 0.5 cm to about 5 cm, about 1 cm to about 5 cm, about 2 cm to about 5 cm, about 1 cm to about 3 cm, about 2 cm to about 10 cm, and about 5 cm to about 15 cm. In another exemplary embodiment, the stent has a conduit or filled conduit shape. In another exemplary embodiment, said first fibrous polymer scaffold is essentially aligned in a direction which is a member selected from longitudinal and circumferential. In another exemplary embodiment, the first fibrous polymer scaffold has a seam. In another exemplary embodiment, the first fibrous polymer scaffold is seamless. In another exemplary embodiment, the first fibrous polymer scaffold is monolithically formed.

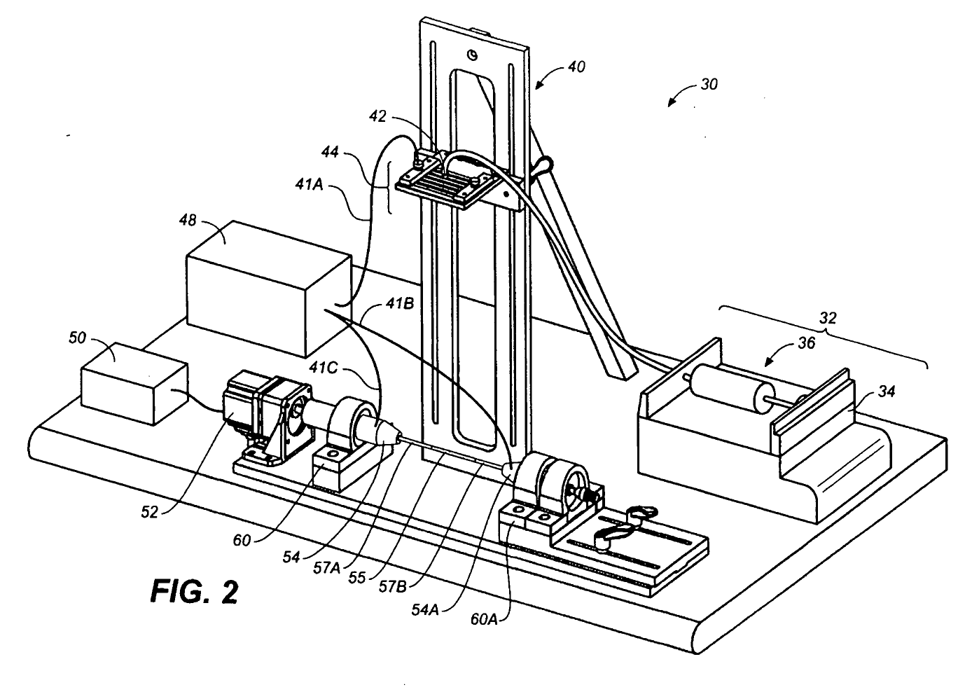

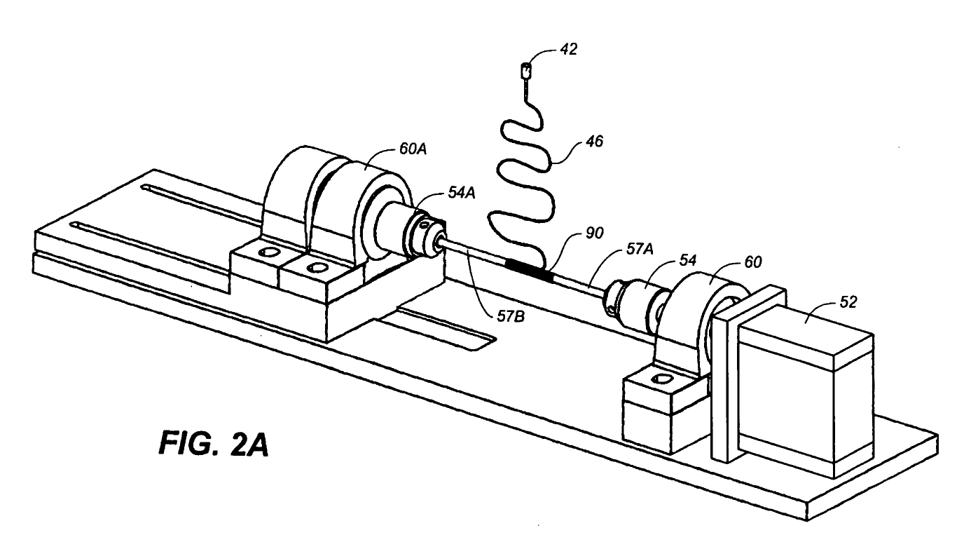

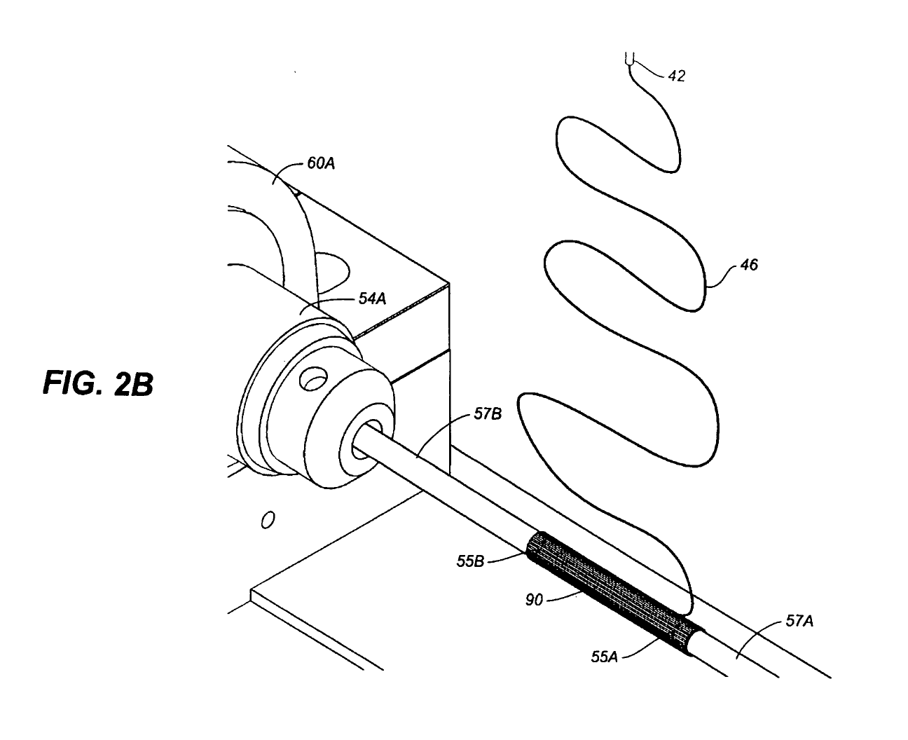

In an exemplary embodiment the stent scaffold is made from a material that will provide the support necessary to hold open the vessel long enough maintain an opening (FIG. 2). In another exemplary embodiment, the stent can be made from one monomer or subunit, for example lactic or polylactic acid, or glycolic or polyglycolic acid can be utilized to form stents made of poly(lactide) (PLA) or poly(L-lactide) (PLLA) or poly(glycolic acid). The scaffold can also be made from more than one polymer thus forming a co-polymer, terpolymer, etc. For example, lactic or polylactic acid and be combined with glycolic acid or polyglycolic acid to form the copolymer poly(glycolide-co-lactide) (PGLA). Other copolymers of use in the invention include poly(ethylene-co-vinyl) alcohol). In an exemplary embodiment, a scaffold comprises a monomer or subunit which is a member selected from polydimethylsiloxane, polylactic acid, polyglycolic acid, polycaprolactone, polyethylene glycol, collagen, elastin, alginate, fibrin, hyaluronic acid. In another exemplary embodiment, a scaffold comprises two different monomers or subunits which are members selected from polydimethylsiloxane, polylactic acid, polyglycolic acid, polycaprolactone, polyethylene glycol, collagen, elastin, alginate, fibrin and hyaluronic acid. In another exemplary embodiment, a scaffold comprises three different monomers or subunits which are members selected from polydimethylsiloxane, polylactic acid, polyglycolic acid, polycaprolactone, polyethylene glycol, collagen, elastin, alginate, fibrin and hyaluronic acid.

Scaffolds can be purchased from commercial sources such as InSitu Technologies Inc, Guidant, DeVax and Boston Scientific.

### Fibrous Polymer Layers

In a first aspect, the invention provides a composition which comprises at least one layer composed of plurality of nanodiameter polymer fibers. A fibrous polymer layer includes a fiber or fibers which can have a range of diameters. In an exemplary embodiment, the average diameter of the fibers in the fibrous polymer layer is in the nanodiameter range. In various embodiments, the diameter is from about 0.1 nanometers to about 50000 nanometers. In another exemplary embodiment, the average diameter of the fibers in the fibrous polymer layer is from about 25 nanometers to about 25,000 nanometers. In an exemplary embodiment, the average diameter of the fibers in the fibrous polymer layer is from about 50 nanometers to about 20,000 nanometers. In an exemplary embodiment, the average diameter of the fibers in the fibrous polymer layer is from about 100 nanometers to about 5,000 nanometers. In an exemplary embodiment, the average diameter of the fibers in the fibrous polymer layer is from about 1,000 nanometers to about 20,000 nanometers. In an exemplary embodiment, the average diameter of the fibers in the fibrous polymer layer is from about 10 nanometers to about 1,000 nanometers. In an exemplary embodiment, the average diameter of the fibers in the fibrous polymer layer is from about 2,000 nanometers to about 10,000 nanometers. In an exemplary embodiment, the average diameter of the fibers in the fibrous polymer layer is from about 0.5 nanometers to about 100 nanometers. In an exemplary embodiment, the average diameter of the fibers in the fibrous polymer layer is from about 0.5 nanometers to about 50 nanometers. In an exemplary embodiment, the average diameter of the fibers in the fibrous polymer layer is from about 1 nanometer to about 35 nanometers. In an exemplary embodiment, the average diameter of the fibers in the fibrous polymer layer is from about 2 nanometers to about 25 nanometers. In an exemplary embodiment, the average diameter of the fibers in the fibrous polymer layer is from about 90 nanometers to about 1,000 nanometers. In an exemplary embodiment, the average diameter of the fibers in the fibrous polymer layer is from about 500 nanometers to about 1,000 nanometers.

In an exemplary embodiment, the fibrous polymer layer is a member selected from a nanofiber polymer layer and a microfiber polymer layer. Microfiber polymer layers have micron-scale features (an average fiber diameter between about 1,000 nanometers and about 50,000 nanometers, and especially between about 1,000 nanometers and about 20,000 nanometers), while nanofiber polymer layers have submicron-scale features (an average fiber diameter between about 10 nanometers and about 1,000 nanometers, and especially between about 50 nanometers and about 1,000 nanometers). Each of these polymer layers can resemble the physical structure at the area of treatment, such as native collagen fibrils or other extracellular matrices.

A variety of polymers from synthetic and/or natural sources can be used to compose these fibrous polymer layers. A fiber can be made from one monomer or subunit. For example, lactic or polylactic acid or glycolic or polyglycolic acid can be utilized to form poly(lactide) (PLA) or poly(L-lactide) (PLLA) nanofibers or poly(glycolide) (PGA) nanofibers. Fibers can also be made from more than one monomer or subunit thus forming a co-polymer, terpolymer, etc. For example, lactic or polylactic acid and be combined with glycolic acid or polyglycolic acid to form the copolymer poly(lactide-co-glycolide) (PLGA). Other copolymers of use in the invention include poly(ethylene-co-vinyl) alcohol). In an exemplary embodiment, a fiber comprises a polymer or subunit which is a member selected from an aliphatic polyester, a polyalkylene oxide, polydimethylsiloxane, polyvinylalcohol, polylysine, collagen, laminin, fibronectin, elastin, alginate, fibrin, hyaluronic acid, proteoglycans, polypeptides and combinations thereof. In another exemplary embodiment, a fiber comprises two different polymers or subunits which are members selected from an aliphatic polyester, a polyalkylene oxide, polydimethylsiloxane, polyvinylalcohol, polylysine, collagen, laminin, fibronectin, elastin, alginate, fibrin, hyaluronic acid, proteoglycans, polypeptides and combinations thereof. In another exemplary embodiment, a fiber comprises three different polymers or subunits which are members selected from an aliphatic polyester, a polyalkylene oxide, polydimethylsiloxane, polyvinylalcohol, polylysine, collagen, laminin, fibronectin, elastin, alginate, fibrin, hyaluronic acid, proteoglycans, polypeptides and combinations thereof. In an exemplary embodiment, the aliphatic polyester is linear or branched. In another exemplary embodiment, the linear aliphatic polyester is a member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In another exemplary embodiment, the aliphatic polyester is branched and comprises at least one member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof which is conjugated to a linker or a biomolecule. In an exemplary embodiment, wherein the polyalkylene oxide is a member selected from polyethylene oxide, polyethylene glycol, polypropylene oxide, polypropylene glycol and combinations thereof.

Still other polymer of use include polyester amides (PEA), polydoxanone (PDO), poly trimethylene carbonate (TMC), polyurethane, including biosorbable polyurethanes, and polyvinyl alcohol.

In some embodiments, the fibrous polymer layer is composed of a single continuous fiber. In other embodiments, the fibrous polymer layer is composed of at least two, three, four, or five fibers. In an exemplary embodiment, the number of fibers in the fibrous polymer layers is a member selected from 2 to 100,000. In an exemplary embodiment, the number of fibers in the fibrous polymer layers is a member selected from 2 to 50,000. In an exemplary embodiment, the number of fibers in the fibrous polymer layers is a member selected from 50,000 to 100,000. In an exemplary embodiment, the number of fibers in the fibrous polymer layers is a member selected from 10 to 20,000. In an exemplary embodiment, the number of fibers in the fibrous polymer layers is a member selected from 15 to 1,000.

The fibrous polymer layer can comprise a fiber of at least one composition. In an exemplary embodiment, the fibrous polymer layer comprises a number of different types of fibers, and this number is a member selected from one, two, three, four, five, six, seven, eight, nine and ten.

In another exemplary embodiment, the fiber or fibers of the fibrous polymer layer are biodegradable. In another exemplary embodiment, the fibers of the fibrous polymer layer comprise biodegradable polymers. In another exemplary embodiment, the biodegradable polymers comprise a monomer which is a member selected from lactic acid and glycolic acid. In another exemplary embodiment, the biodegradable polymers are poly(lactic acid), poly(glycolic acid) or a copolymer thereof. Preferred biodegradable polymers are those which are approved by the FDA for clinical use, such as poly(lactic acid) and poly(glycolic acid). In another exemplary embodiment, biodegradable polymer layers of the invention can be used to guide the morphogenesis of engineered tissue and gradually degrade after the assembly of the tissue. The degradation rate of the polymers can be tailored by one of skill in the art to match the tissue generation rate. For example, if a polymer that biodegrades quickly is desired, an approximately 50:50 PLGA combination can be selected. Additional ways to increase polymer layer biodegradability can involve selecting a more hydrophilic copolymer (for example, polyethylene glycol), decreasing the molecular weight of the polymer, as higher molecular weight often means a slower degradation rate, and changing the porosity or fiber density, as higher porosity and lower fiber density often lead to more water absorption and faster degradation. In another exemplary embodiment, the tissue is vascular tissue.

### Methods of Making a Fibrous Polymer Layer

The polymer layers of the invention can be produced in a variety of ways. In an exemplary embodiment, the polymer layer can be produced by electrospinning. Electrospinning is an atomization process of a conducting fluid which exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material can be collected as an interconnected web containing relatively fine, i.e. small diameter, fibers. The resulting films (or membranes) from these small diameter fibers have very large surface area to volume ratios and small pore sizes. A detailed description of electrospinning apparatus is provided in Zong, et al., Polymer, 43(16):4403-4412 (2002); Rosen et al., Ann Plast Surg., 25:375-87 (1990) Kim, K., Biomaterials 2003, 24, (27), 4977-85; Zong, X., Biomaterials 2005, 26, (26), 5330-8. After electrospinninng, extrusion and molding can be utilized to further fashion the polymers. To modulate fiber organization into aligned fibrous polymer layers, the use of patterned electrodes, wire drum collectors, or post-processing methods such as uniaxial stretching has been successful. Zong, X., Biomaterials 2005, 26, (26), 5330-8; Katta, P., Nano Lett 2004, 4, (11), 2215-2218; Li, D., Nano Lett 2005, 5, (5), 913-6.

The polymer solution can be produced in one of several ways. One method involves polymerizing the monomers and dissolving the subsequent polymer in appropriate solvents. This process can be accomplished in a syringe assembly or it can be subsequently loaded into a syringe assembly. Another method involves purchasing commercially available polymer solutions or commercially available polymers and dissolving them to create polymer solutions. For example, PLLA can be purchased from DuPont (Wilmington, Del.), poly(lactide-co-glycolide) can be purchased from Ethicon (Somerville, N.J.) and Birmingham Polymers (Birmingham, Ala.), Sigma-Aldrich (St. Louis, Mo.) and Polysciences (Warrington, Pa.). Other manufacturers include Lactel Absorbable Polymers (Pelham, Ala.). Additional polymer layer components of the invention, such as cells and biomolecules, are also commercially available from suppliers such as Invitrogen (San Diego, Calif.), Cambrex (Walkersville, Md.), Sigma-Aldrich, Peprotech (Rocky Hill, N.J.), R&D Systems (Minneapolis, Minn.), ATCC (Manassas, Va.), Pierce Biotechnology (Rockford, Ill.).

The polymer used to form the polymer layer is first dissolved in a solvent. The solvent can be any solvent which is capable of dissolving the polymer monomers and/or subunits and providing a polymer solution capable of conducting and being electrospun. Typical solvents include a solvent selected from N,N-Dimethyl formamide (DMF), tetrahydrofuran (THF), methylene chloride, dioxane, ethanol, hexafluoroisopropanol (HFIP), chloroform, water and combinations thereof.

The polymer solution can optionally contain a salt which creates an excess charge effect to facilitate the electrospinning process. Examples of suitable salts include NaCl, KH₂PO₄, K₂HPO₄, KIO₃, KCl, MgSO₄, MgCl₂, NaHCO₃, CaCl₂ or mixtures of these salts.

The polymer solution forming the conducting fluid will preferably have a polymer concentration in the range of about 1 to about 80 wt %, more preferably about 8 to about 60 wt %. The conducting fluid will preferably have a viscosity in the range of about 50 to about 2000 mPa×s, more preferably about 200 to about 700 mPa×s.

The electric field created in the electrospinning process will preferably be in the range of about 5 to about 100 kilovolts (kV), more preferably about 10 to about 50 kV. The feed rate of the conducting fluid to the spinneret (or electrode) will preferably be in the range of about 0.1 to about 1000 microliters/min, more preferably about 1 to about 250 microliters/min.

The single or multiple spinnerets sit on a platform which is capable of being adjusted, varying the distance between the platform and the grounded collector substrate. The distance can be any distance which allows the solvent to essentially completely evaporate prior to the contact of the polymer with the grounded collector substrate. In an exemplary embodiment, this distance can vary from 1 cm to 25 cm. Increasing the distance between the grounded collector substrate and the platform generally produces thinner fibers.

In electrospinning cases where a rotating mandrel is required, the mandrel is mechanically attached to a motor, often through a drill chuck. In an exemplary embodiment, the motor rotates the mandrel at a speed of between about 1 revolution per minute (rpm) to about 500 rpm. In an exemplary embodiment, the motor rotation speed of between about 200 rpm to about 500 rpm. In another exemplary embodiment, the motor rotation speed of between about 1 rpm to about 100 rpm.

Additional embodiments or modifications to the electrospinning process are described herein.

### Variation of Electrical/Mechanical Properties of Conducting Fluid

The properties of the material produced by electrospinning is affected by the electric and mechanical properties of the conducting fluid. The conductivity of the polymer solution can be drastically changed by adding ionic inorganic/organic compounds. The magneto-hydrodynamic properties of the polymer solution can depend on a combination of physical and mechanical properties, (e.g., surface tension, viscosity and viscoelastic behavior of the fluid) and electrical properties (e.g., charge density and polarizability of the fluid). For example, by adding a surfactant to the polymer solution, the fluid surface tension can be reduced, so that the electrostatic fields can influence the jet shape and the jet flow over a wider range of conditions. By coupling a syringe pump that can control the flow rate either at constant pressure or at constant flow rate, the effect of viscosity of the conducting fluid can be alleviated.

### Electrode Design

In another embodiment for producing plurality of nanodiameter polymer fibers layers of use in the present invention, the jet formation process during electrospinning is further refined to provide better control over fiber size. Instead of merely providing a charged spinneret and a ground plate, as discussed above, a positively charged spinneret is still responsible for the formation of the polymer solution droplet and a plate electrode with a small exit hole in the center is responsible for the formation of the jet stream. This exit hole will provide the means to let the jet stream pass through the plate electrode. Thus, if the polymer droplet on the positively charged spinneret has a typical dimension of 2-3 mm and the plate electrode is placed at a distance of about 10 mm from the spinneret, a reasonable electrostatic potential can be developed. By varying the electric potential of the spinneret, the jet formation can be controlled and adjusted. Such an electrode configuration reduces the required applied potential on the spinneret from typically about 15 kilovolts (kV) down to typically about 1.5 to 2 kV (relative to the ground plate potential). The exact spinneret potential required for stable jet formation will depend on the electrical/mechanical properties of the specific conducting fluid and is readily determined by one of skill in the art.

### Control of Jet Acceleration and Transportation

In another preferred embodiment for producing plurality of nanodiameter polymer fiber layers of the present invention, the jet stream flight is also precisely controlled. The jet stream passing through the plate electrode exit hole is positively charged. Although this stream has a tendency to straighten itself during flight, without external electric field confinement the jet will soon become unstable in its trajectory. In other words, the charged stream becomes defocused, resulting in loss of control over the microscopic and macroscopic properties of the fluid. This instability can be removed by using a carefully designed probe electrode immediately after the plate electrode and a series of (equally) spaced plate electrodes. The electrode assembly (or composite electrode), i.e., the probe electrode and the plate electrodes, can create a uniform distribution of electrostatic potential along the (straight) flight path. The acceleration potential is formed by placing the base potential of the spinneret at about +20 to +30 kV above the target (at ground potential) while the electrostatic potential of the probe electrode can be adjusted to slightly below the plate electrode base potential. The composite electrodes are capable of delivering the jet stream to a desired target area. The composite electrode can also be utilized to manipulate the jet stream. By changing the electrostatic potential, the jet stream acceleration is altered, resulting in varying the diameter of the formed polymer fiber. This electrostatic potential variation changes the jet stream stability, and therefore, corresponding changes in the composite electrode can be used to stabilize the new jet stream. As understood by those of skill in the art, such a procedure can be used to fine-tune and to change the fiber diameter during the electrospinning process.

### Jet Manipulation

In yet another embodiment, the jet stream can be focused by using an “Alternating Gradient” (AG) technique, widely used in the accelerator technology of high-energy physics. The basic idea is to use two pairs of electrostatic quadrupole lenses. The second lens has the same geometric arrangement as the first lens with a reversed (alternate) electric gradient. The positively charged jet stream will be focused, for example, in the xz plane after the first lens and then be refocused in the yz plane after the second lens. It is noted that the z-direction represents the direction of the initial flight path. By applying an additional triangle-shaped waveform to the potential on one of the pairs of the quadrupole, the jet can be swept across the target area, allowing the control of the direction of the jet stream. Furthermore, with varying waveform of the ‘sweep’ potential, a desired pattern on the target can be formed.

Electrospinning Apparatus with Mandrels

In another aspect, the invention includes a method to electrospin a layer of a plurality of nanodiameter polymer fibers (e.g., aligned fibrous polymer layers) for use in stents on a rotating mandrel. These fibrous polymer layers can be aligned in any orientation desired by the user. In an exemplary embodiment, the layers are aligned in an essentially longitudinal or essentially circumferential direction. The fibrous polymer layer created by this method can either have a seam or they can be seamless. In an exemplary embodiment, the fibrous polymer layers are seamless. In another exemplary embodiment, the fibrous polymer layers are seamless along an axis essentially parallel to the longitudinal axis of the polymer scaffolds.

In one embodiment, the stent scaffold is utilized as the mandrel.

In another exemplary embodiment, the scaffold is composed of a fibrous polymer layer configured as a conduit having an intraluminal space. In this embodiment, the scaffolds (e.g., seamless scaffolds) have an unaligned fiber orientation. In another exemplary embodiment, the fibers of the scaffolds are aligned. In another exemplary embodiment, the scaffolds and have essentially longitudinally aligned fibers. In an exemplary embodiment, the mandrel is attached to a motor assembly that is capable of rotating the mandrel about its longitudinal axis. In the electrospinning apparatus, the rotating mandrel is grounded and placed below a spinneret. A polymer solution is delivered to the tip of the spinneret and is charged by a power supply. The electrical field created between the spinneret and the mandrel induces the charged polymer solution at the tip of the spinneret to form a jet. The jet sprays toward the mandrel. The polymer contacts one conducting region of the mandrel and then contacts a second conducting region of the mandrel, depositing the fiber across a non-conducting region or air gap of the mandrel. This results in the formation of aligned fibers deposited on the non-conducting region or in the air gap. By rotating the mandrel, the result is an evenly applied layer of aligned fibers. The deposited fibrous layers conform to the shape of the mandrel or the air gap between the mandrels, thus forming a sheet in some instances, a conduit in some instances or a rod in other instances. The fibers comprising the sheet or conduit will be aligned along the length of the conduit, thus forming a sheet, conduit or rod with longitudinally aligned fibers. In an exemplary embodiment, the conduit or rod will be seamless. In an exemplary embodiment, the conduit or rod will be seamless along an axis that is essentially parallel to the long axis of the conduit or rod.

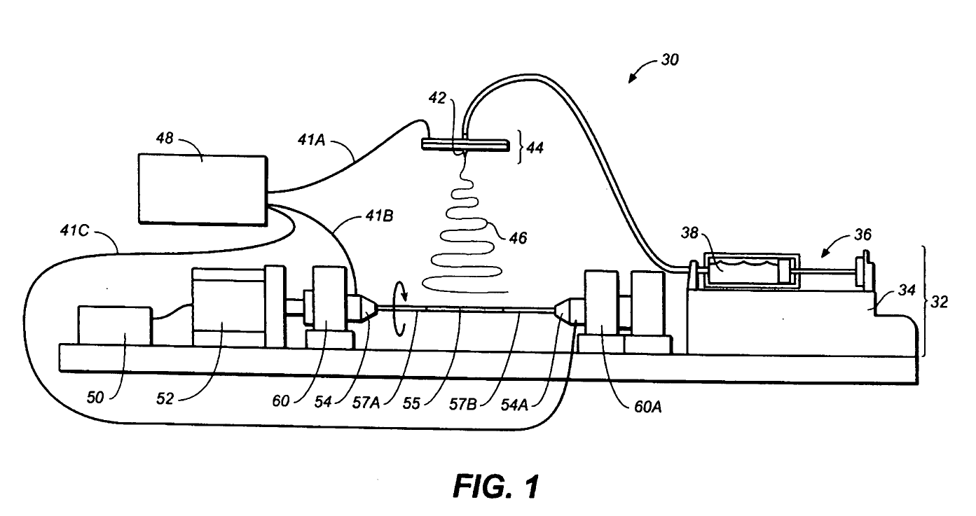

In one embodiment, the fibrous polymers of use in the invention can be created on a mandrel with at least two conducting regions and at least one non-conducting region. Such a mandrel can be designed in a number of ways; an exemplary depiction of the mandrel is provided in FIG. 3B and an exemplary depiction of the mandrel as part of an apparatus for producing sheets and/or conduits of the invention is described in FIGS. 1, 2, 2A, and 2B. In an exemplary embodiment, the electrically conducting material is a metal. In another exemplary embodiment, the metal is a member selected from steel and aluminum. In one instance, a region of a conducting mandrel can be covered with a non-electrically conducting material. An exemplary cross section of this mandrel is provided in FIG. 3D. In an exemplary embodiment, the non-electrically conducting material is a member selected from tape, electrical tape, teflon and plastic. In another instance, a mandrel can be produced that has at least three sections, a non-electrically conducting region which interconnects two conducting mandrel regions. In another instance, a non-electrically conducting region is a discrete portion extending between two conducting mandrel regions. An exemplary cross section of this mandrel is provided in FIG. 3C.

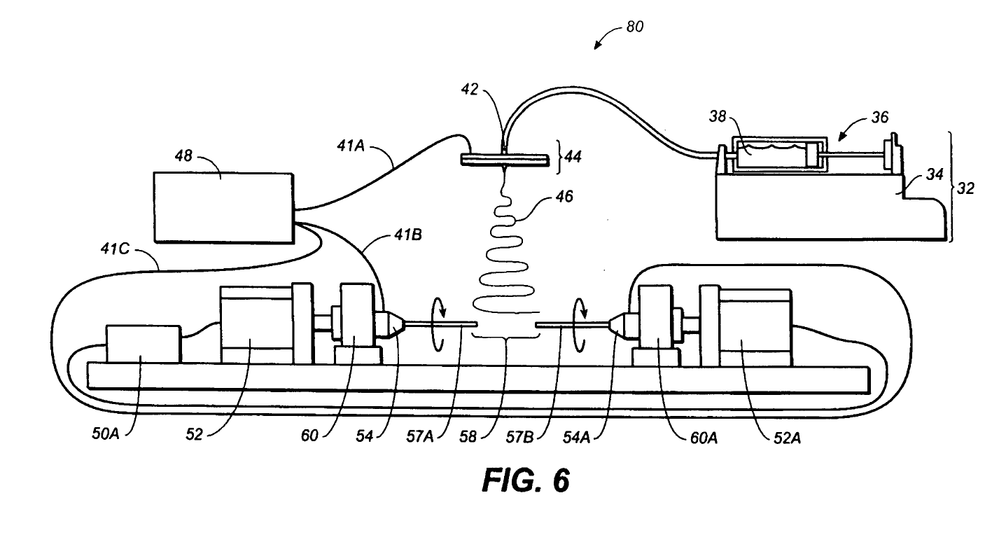

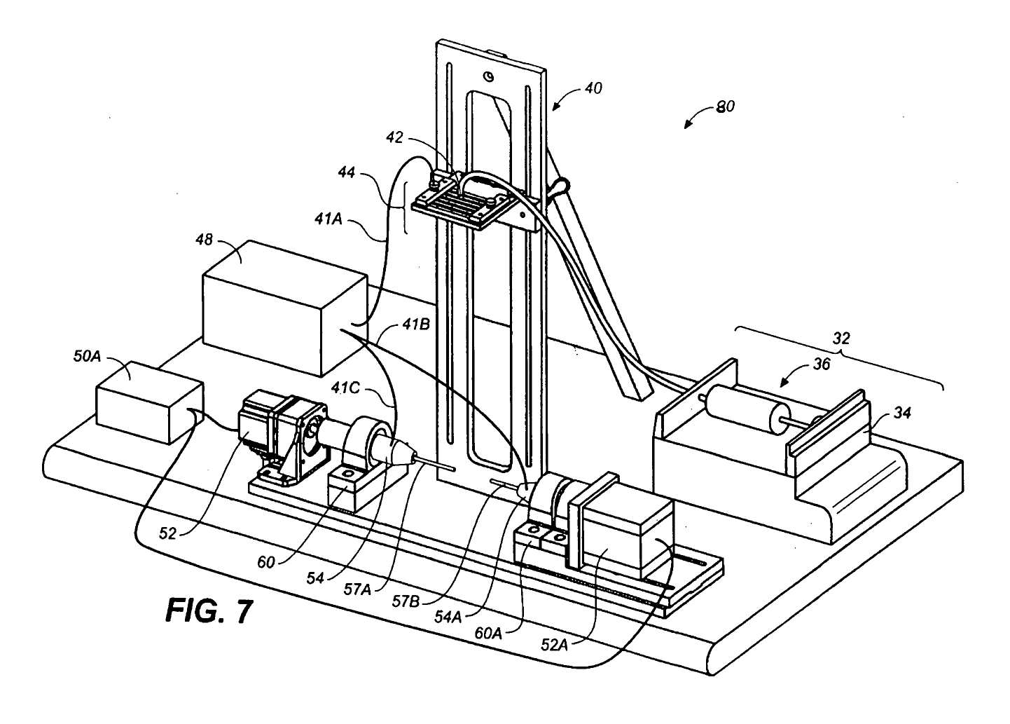

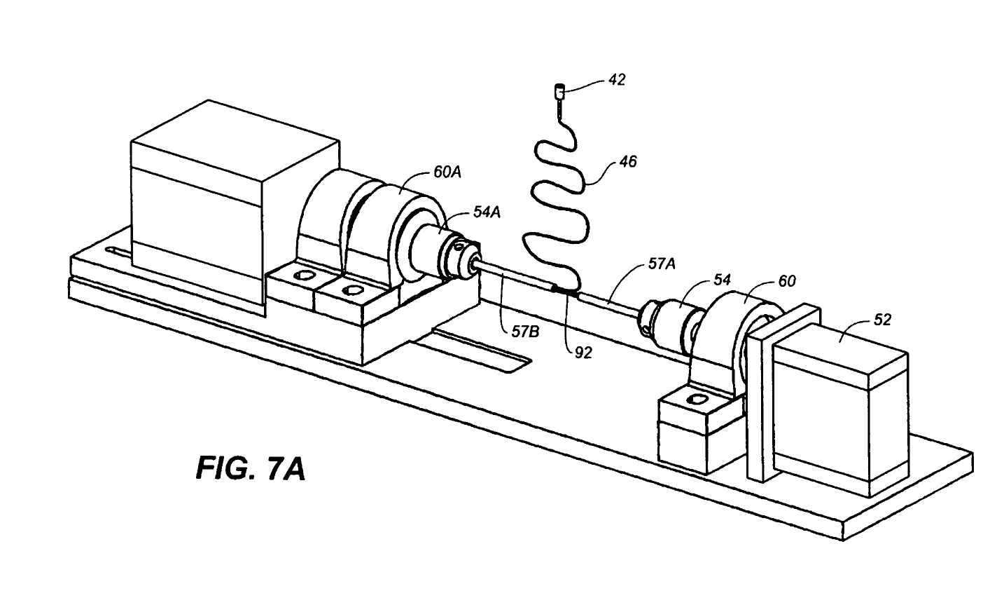

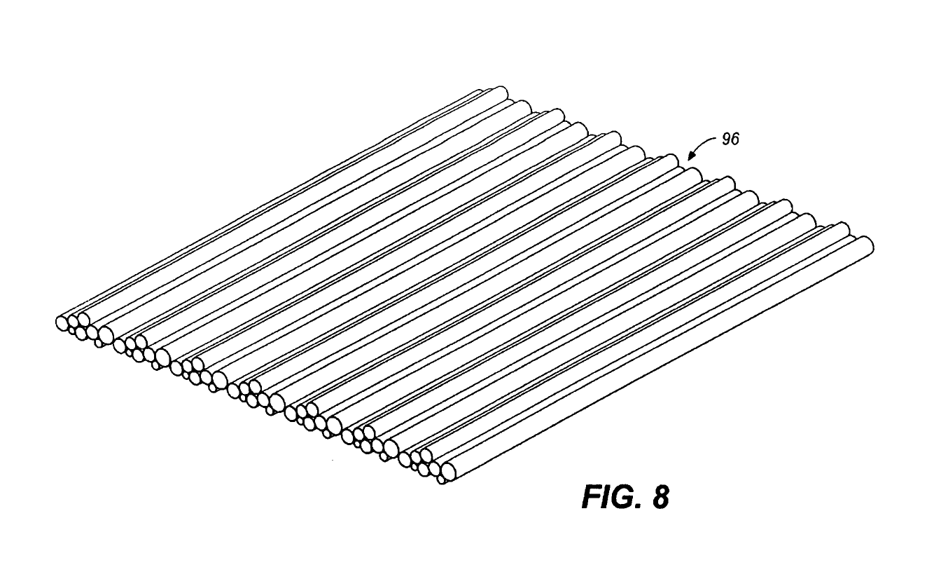

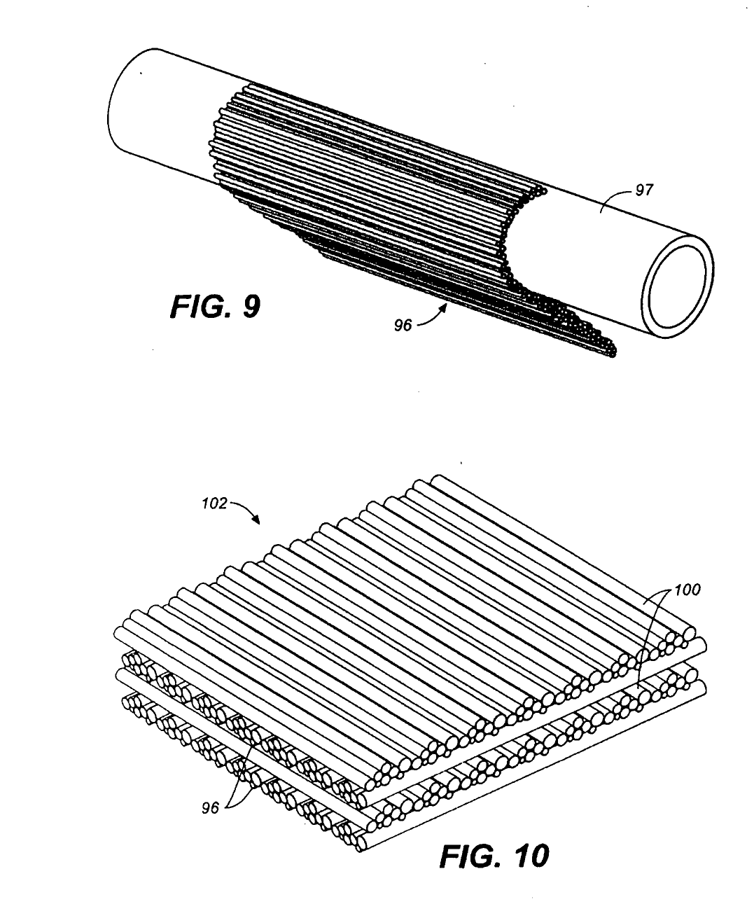

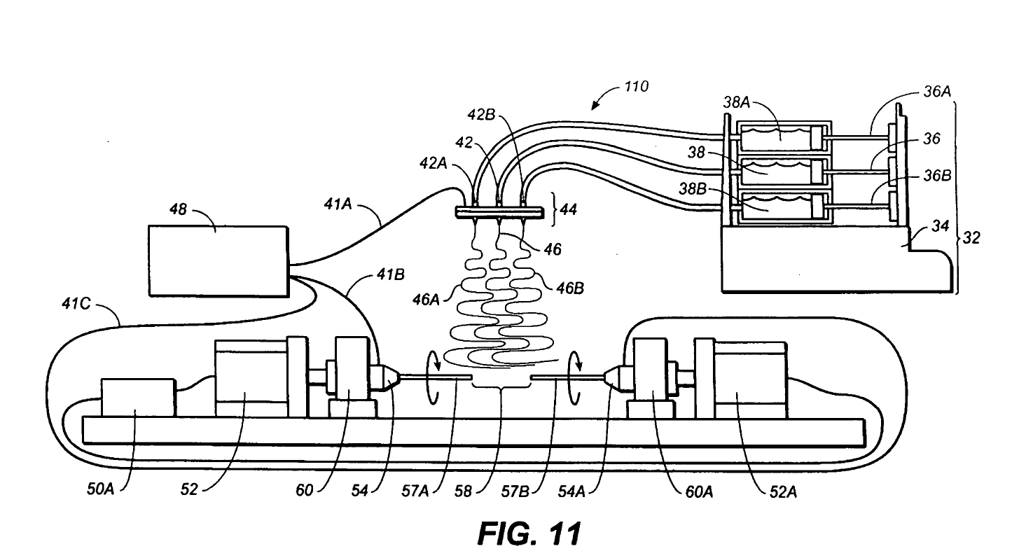

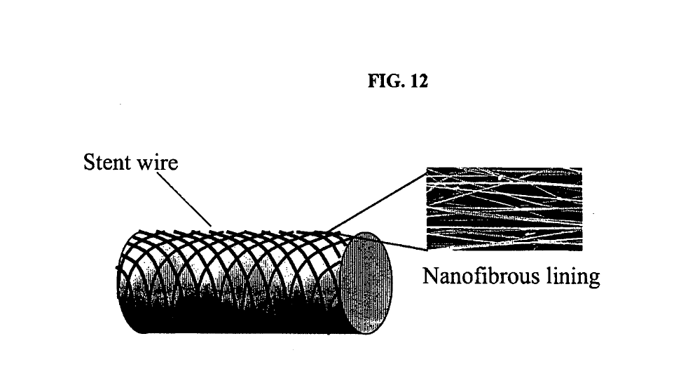

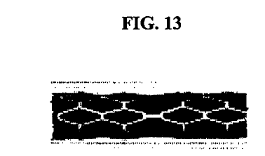

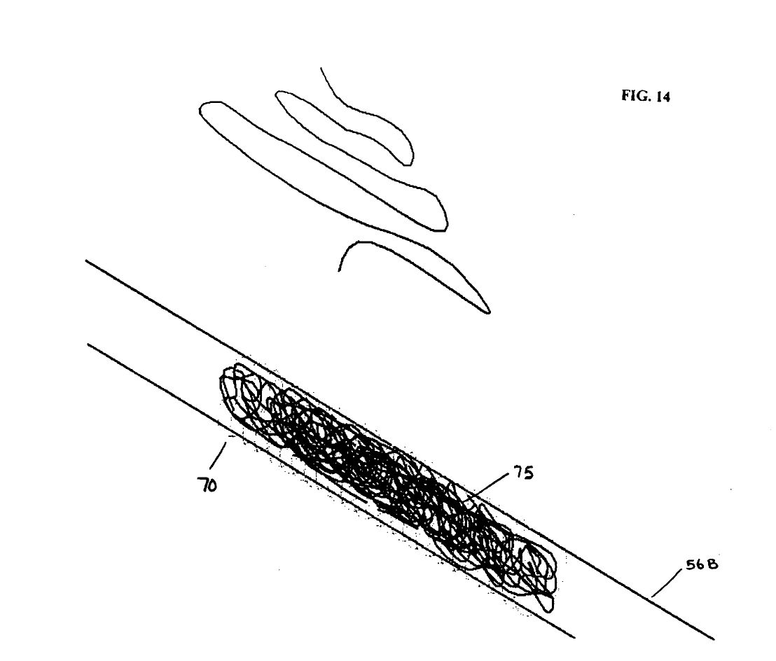

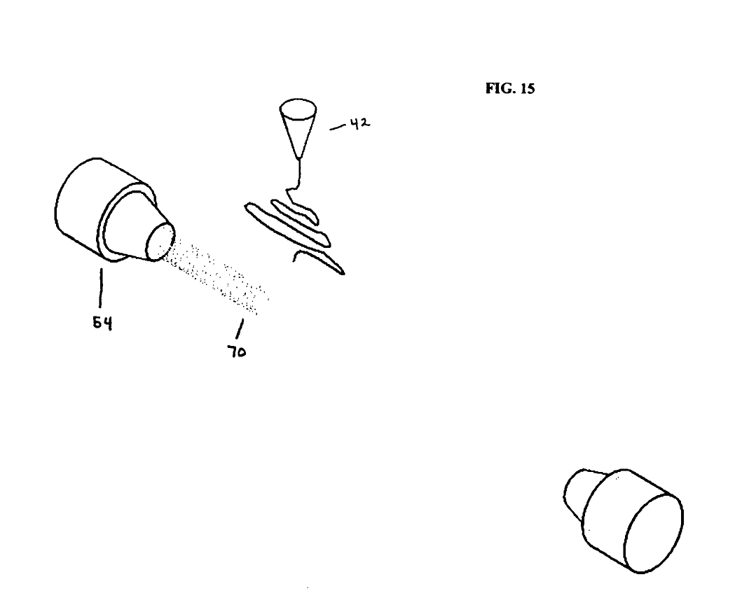

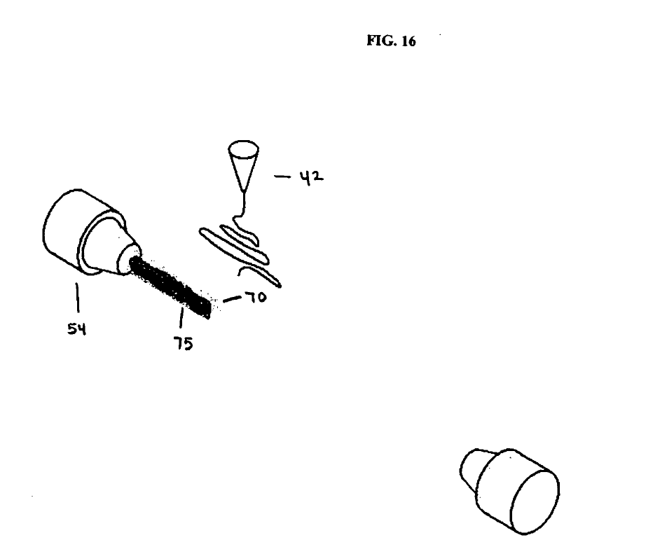

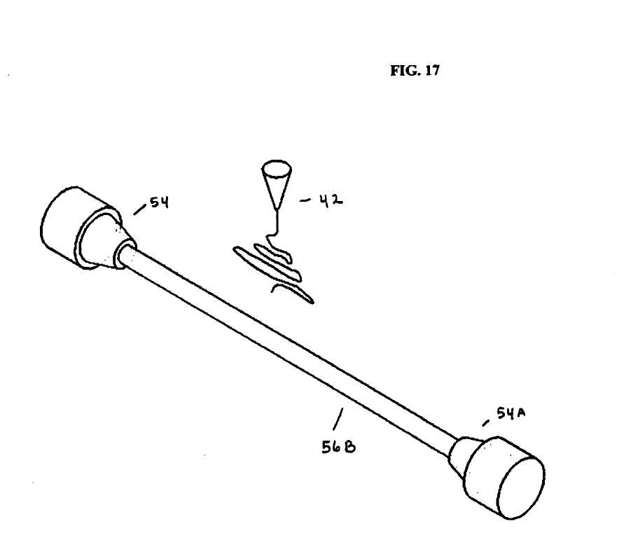

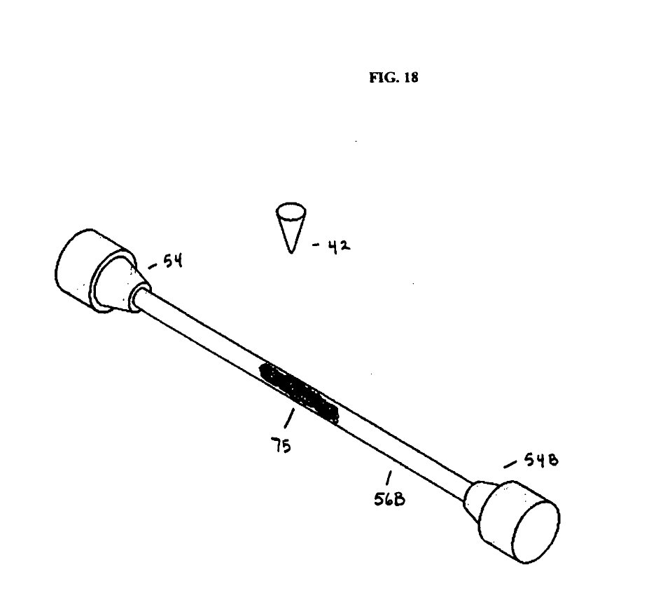

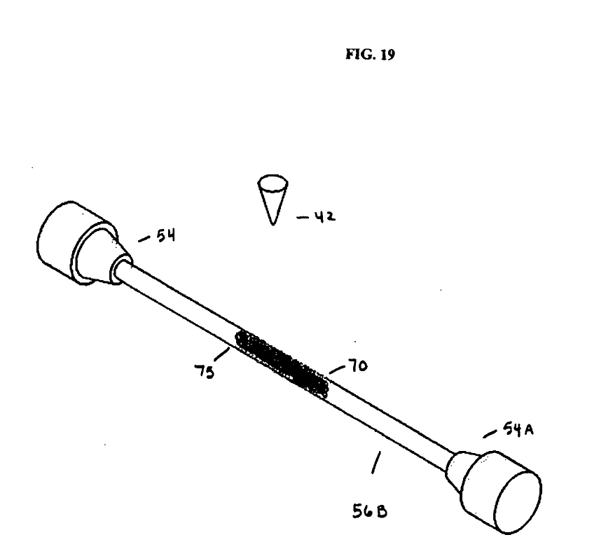

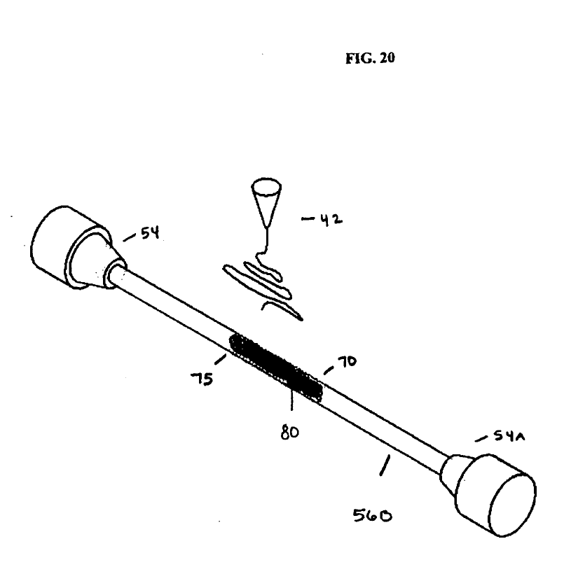

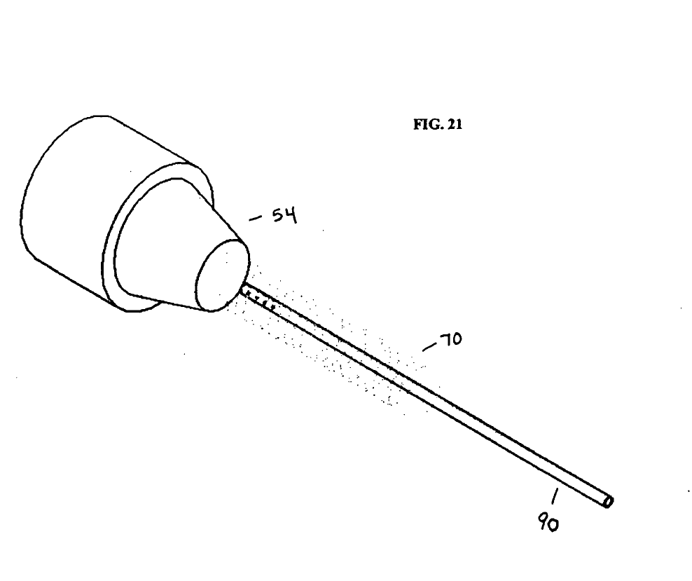

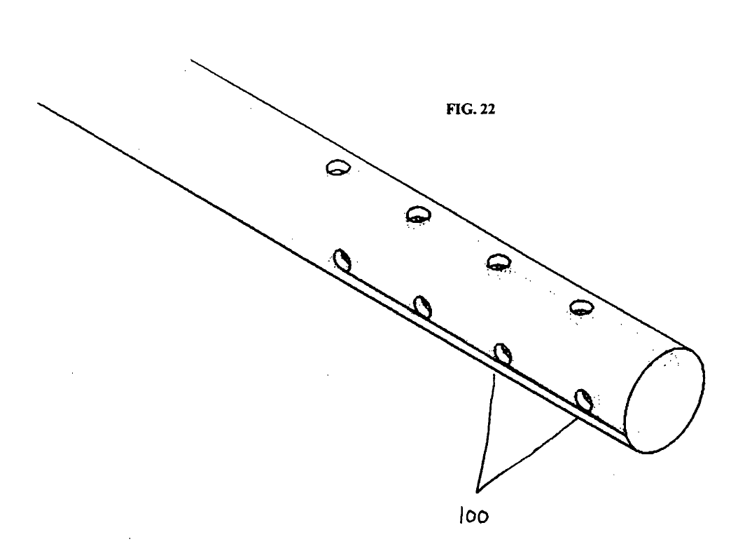

In one embodiment, the fibrous polymers of use in the invention can be created on a mandrel with a first conducting region, a second conducting region, and an air gap between the first conducting region and the second conducting region. Such a mandrel can be designed in a number of ways; an exemplary depiction of the mandrel is provided in FIG. 3E and an exemplary depiction of the mandrel as part of an apparatus for producing rods of the invention is described in FIGS. 6, 7, and 7A. An embodiment with multiple spinnerets is provided is described in FIG. 11. In an exemplary embodiment, the electrically conducting material is a metal. In another exemplary embodiment, the metal is a member selected from steel and aluminum. In an exemplary embodiment, each conducting region of the mandrel is aligned with the other. In an exemplary embodiment, each conducting region of the mandrel is attached to assemblies that are capable of rotating at the same speed. This can be accomplished by attaching motor assemblies to each conducting region of the mandrel and ensuring that each motor runs at the same speed. This can also be accomplished by ensuring that each conducting region of the mandrel is connected to the same motor.

After electrospinning is complete, the stents, or stent scaffolds of the invention are removed from the mandrel. For sheet polymer scaffolds, the sheet can be peeled away from the mandrel. For conduit polymer scaffolds, the mandrel can be taken out of the motor assembly and the conduit can then be removed. In some embodiments, removal can also be accomplished by disconnecting the mandrel in the middle or also by cutting the conduit. In some instances, the lengths of the deposited fibers will not be equal, resulting in jagged edges at the end or ends of the polymer scaffold. Optionally, the ends of the polymer scaffolds can be cut in order to create polymer scaffolds with lengths of essentially the same size. This cutting can occur when the polymer scaffold is on the mandrel, or after it has been removed from the mandrel. Stents can be slid off the mandrel, or if the stent scaffold itself serves as the mandrel, the stent is simply removed from the electrospinning device.

The characteristics of the polymer materials described herein can be changed by altering various parameters. For example, there are several methods which either alone or in combination can decrease the average diameter of the fibers in the fibrous polymer scaffold. One method is to add more salt to the polymer solution. Using a more polar solvent in the polymer solution also tends to decrease the average fiber diameter, as does increasing the distance between the spinneret and the mandrel. Additional methods of decreasing the scaffold diameter include increasing the apparatus voltage and increasing the polymer concentration.

Multi-layered polymer scaffolds and stents can be formed by the methods described herein by completing several mandrel rotations. For example, a multilayered conduit or stent can be formed by completing several mandrel rotations. Polymer scaffolds or stents with more than one type of polymer layer can also be produced. In an exemplary embodiment, the circumferential alignment of the fibers can also be adjusted or varied by altering the speed by which the mandrel rotates. Thus a polymer scaffold with an inner longitudinally aligned layer and an outer circumferentially aligned layer can be produced. In order to fabricate a multi-layered hollow conduit scaffold with each layer having specific alignment, various mandrels and rotation speeds may be used. In an exemplary embodiment, a hollow conduit shaped fibrous scaffold is produced with a luminal layer composed of longitudinally aligned fibers and an outer layer with circumferentially aligned fibers. One method to produce such a scaffold involves using a mandrel with a non-conducting region as described previously. The mandrel is rotated at a slow speed allowing for the formation of a conduit shaped fibrous scaffold composed of longitudinally aligned fibers. The rotation speed of the mandrel is then increased, which causes the electrospun fibers to align in a circumferential direction around the longitudinally aligned fibrous conduit. In another exemplary embodiment, the inner layer is aligned longitudinally while the outer layer is composed of randomly aligned fibers. This can be achieved using the same setup as described previously except when forming the outer layer, the mandrel is rotated at an intermediate speed that prevents both longitudinal and circumferential alignment of fibers.

### Alignment of the Polymer

The polymer scaffolds and stents of the invention can have an aligned orientation or a random orientation. In one embodiment, an aligned orientation is charaterized by having at least 50% of the fibers comprising the polymer scaffold oriented along an average axis of alignment. As will be appreciated by those of skill in the art, the stents of the invention include one or more layer formed from a plurality of nanodiameter polymer fibers which can be similarly aligned or random. For clarity, the following discussion focuses on the polymeric scaffolds, however, it is equally applicable to the layered components of the stents of the invention.

In an exemplary embodiment, the scaffold or layer(s) has an alignment which is a member selected from essentially longitudinal, essentially circumferential, and ‘criss-cross’. A longitudinal alignment is present when the fibers are aligned in the direction of the long axis of the conduit, filled conduit or rod shaped stent scaffolds. A circumferential alignment is present when the fibers are aligned along the short axis of the stent scaffold. A criss-cross alignment is present when the fibers of one polymer scaffold in the composition are aligned in such a manner that the average alignment axis of a first polymer scaffold is at an angle relative to the average alignment axis of a second polymer scaffold which is adjacent to the first polymer scaffold. A longitudinally aligned or circumferentially aligned polymer scaffold can have more than one layer of fibers. A criss-cross aligned polymer scaffold requires more than one layer of fibers.

In another exemplary embodiment, the polymer fibers can have a standard deviation from the central axis of the fiber bundle, or an axis of the scaffold. In an exemplary embodiment, the standard deviation of the fiber is a member selected from between about 0° and about 10, between about 0° and about 3°, between about 0° and about 5°, between about 0° and about 10°, between about 0° and about 15°, between about 0° and about 20°, and between about 0° and about 30°.

In another exemplary embodiment, the stents and stent scaffolds of the invention (such as polymer scaffolds) can comprise biodegradable polymers. These materials can be used to guide the morphogenesis of other types of tissues with anisotropic structure, e.g., blood vessels. These aligned, biodegradable materials of the invention can also be used in the development of three-dimensional tissues. Using electrospun biodegradable fibrous polymer scaffolds, three-dimensional constructs of vascular tissue can be created.

In an exemplary embodiment, the compositions described herein can comprise more than one type of scaffold, e.g. a polymer scaffold combined with a metallic scaffold, or two or more scaffolds of different morphology or composition. When two or more polymer scaffolds are used, each of those polymer scaffolds can have an alignment which is the same or different from the other polymer scaffold or scaffolds in the composition.

In an exemplary embodiment, the composition comprises two polymer scaffolds. The first polymer scaffold has the shape of a conduit and is longitudinally aligned. The second polymer scaffold surrounds the exterior of the first polymer scaffold and has an orientation which is a member selected from random, circumferential, criss-cross, and longitudinal. In an exemplary embodiment, the orientation of the second polymer scaffold is a member selected from random and circumferential.

### Shapes of the Polymer Scaffolds/Methods of Makin, the Polymer Scaffolds

The polymer scaffolds of the invention can be formed into a variety of shapes, depending on the nature of the problem to be solved.

In an exemplary embodiment, the polymer scaffold has the shape of a sheet or membrane. Polymer scaffold membranes can be made through electrospinning. The individual fibers within the membrane can be aligned either during electrospinning using a rotating drum as a collector or after by mechanical uniaxial stretching.

In another exemplary embodiment, the polymer scaffold has the shape of a ‘criss-cross’ sheet. To form a criss-cross sheet, layers of aligned polymer sheets or membranes can be arranged in relation to each other, at an angle which is a member selected from greater than 20 degrees but less than 160 degrees, greater than 30 degrees but less than 150 degrees, greater than 40 degrees but less than 140 degrees, greater than 50 degrees but less than 130 degrees, greater than 60 degrees but less than 120 degrees, greater than 70 degrees but less than 110 degrees, and greater than 80 degrees but less than 100 degrees.

There are a variety of ways to make a ‘criss-cross’ sheet. In one exemplary embodiment, a rotating metal drum collector is used that does not contain a non-conducting region. An aligned layer of fibers is created on the drum, which is then peeled off the drum. The aligned layer is rotated 90 degrees and then placed back on the drum. Next an additional layer of electrospun fibers is added while the drum rotates at a high speed. Additional criss-cross layers can be added by repeating these steps. In another exemplary embodiment, a drum is used that has a non-conducting region. Here, the drum is rotated slowly for a first period of time so the fibers deposit and align longitudinally on the non-conducting section. Then the drum is spun fast so the fibers are forced to align circumferentially. Additional criss-cross layers can be added by repeating these steps.

### Conduit

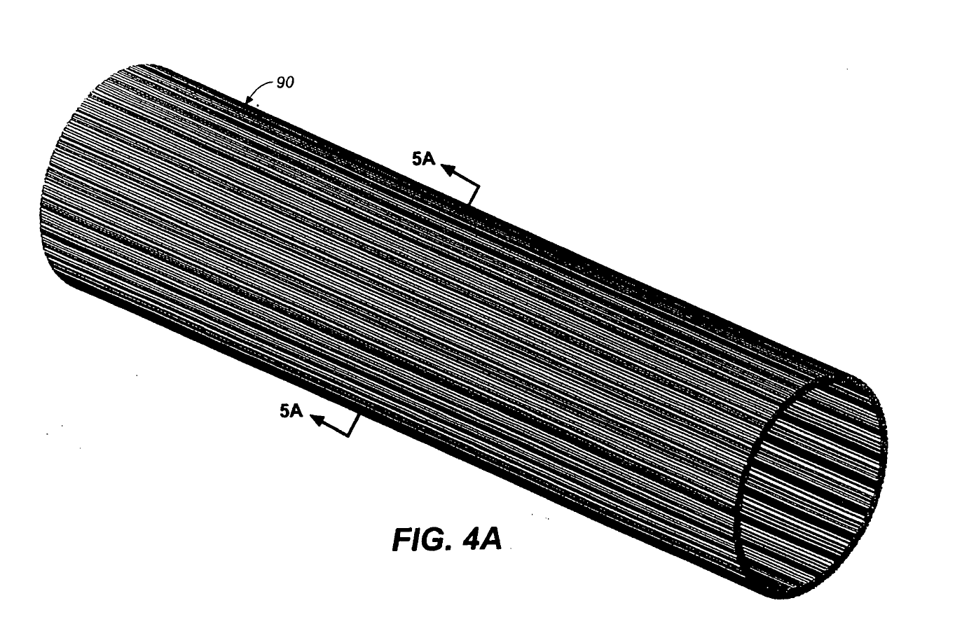

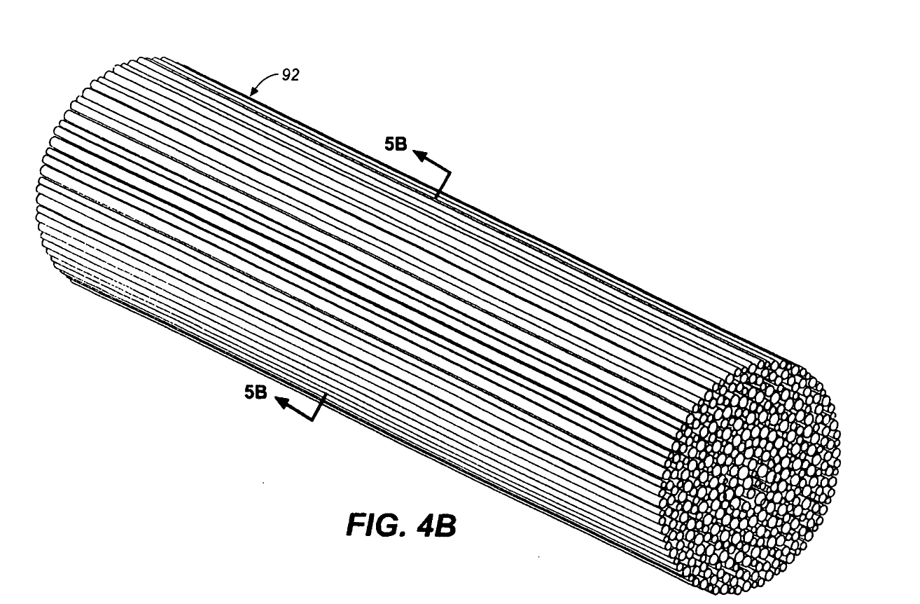

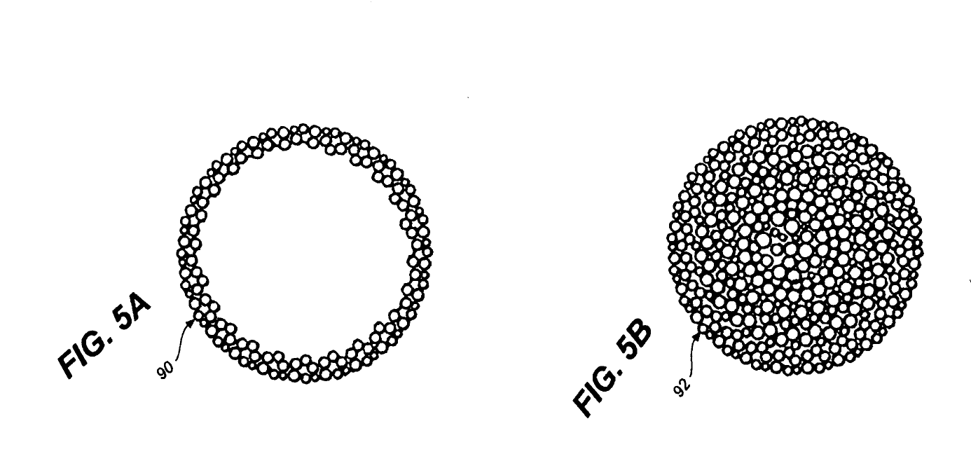

In another exemplary embodiment, the scaffold, e.g., the polymer scaffold has the shape of a conduit. An exemplary depiction of a conduit is provided in FIG. 4A and an exemplary depiction of a cross-sectional view of the conduit is provided in FIG. 5A. A conduit can have a variety of sizes, depending on its length, as well as its inner diameter and outer diameters. In an exemplary embodiment, the interior space of the conduit is essentially free of a fibrous polymer scaffold. These parameters can be varied to accommodate, for example, various tissue sizes and applications. In an exemplary embodiment, the conduit wall is comprised of aligned fibers. In another exemplary embodiment, the fibers are longitudinally aligned or circumferentially aligned. In another exemplary embodiment, the conduit has a seam. In another exemplary embodiment, the seam of the conduit is essentially parallel to the longitudinal axis of the conduit. In another exemplary embodiment, the conduit is seamless. In another exemplary embodiment, the conduit is essentially seamless parallel to the longitudinal axis of the conduit. In another exemplary embodiment, the inner wall of the conduit consists of a layer of longitudinally aligned fibers while the outer wall of the conduit is composed of unaligned fibers. In another exemplary embodiment, the conduit is seamless parallel to the longitudinal axis of the conduit, and the inner wall of the conduit consists of a layer of longitudinally aligned fibers while the outer wall of the conduit is composed of unaligned fibers. The conduit defined in this instance is designed to display greater structural integrity due to the presence of randomly oriented fibers as an outer sheath. In another exemplary embodiment, the inner wall of the conduit is composed of unaligned randomly oriented fibers while the outer wall of the conduit is composed of a layer of longitudinally aligned fibers. In another exemplary embodiment, this conduit is seamless parallel to the longitudinal axis of the conduit. In another exemplary embodiment, the inner wall of the conduit is composed of longitudinally aligned fibers while the outer wall of the conduit is composed of circumferentially aligned fibers. In another exemplary embodiment, this conduit is seamless along an axis essentially parallel to the longitudinal axis of the conduit.

The conduits described herein can be produced in a number of ways. In an exemplary embodiment, the conduit is not electrospun. In another exemplary embodiment, the conduit is composed of random unoriented fibers or a random unoriented polymer scaffold.

In an exemplary embodiment, a fibrous polymer scaffold sheet is rolled to fabricate a conduit with a seam. First, a fibrous polymer scaffold sheet is/are electrospun. The fibers comprising the sheet can be aligned during electrospinning. Some methods which produce aligned electrospun fibers include the use either of a rotating drum as a grounded collector substrate or by using a mandrel described herein. In an exemplary embodiment, the mandrel is mandrel 56A. The fibers comprising the sheet can also be aligned after electrospinning by mechanical uniaxial stretching. The aligned fibrous polymer scaffold sheet is then rolled around a mandrel to form a conduit. The mandrel can either be removed either before or after the conduit is fastened. In an exemplary embodiment, the two ends of the sheet which are parallel to the longitudinal axis of the polymer scaffold are then fastened together to produce a longitudinally aligned seamed conduit. In an exemplary embodiment, the sheet is rolled around the mandrel more than once and one end of the sheet is fastened to a part of the conduit to create a longitudinally aligned seamed conduit. The fastening can be accomplished by annealing (heat), adhesion or by sutures. Examples of adhesion involve solvents or biological adhesives such as fibrin sealant and collagen gels.

Reference will now be made in detail to several embodiments of the invention, examples of which are illustrated in the accompanying drawings. While the invention will be described in conjunction with the subsequent embodiments, it will be understood that they are not intended to limit the invention to those embodiments. On the contrary, the invention is intended to cover alternatives, modifications and equivalents, which may be included within the spirit and scope of the invention as defined by the appended claims.

In another exemplary embodiment, the invention provides a seamless conduit for use in a stent. FIG. 1 refers to an electrospinning apparatus 30 for producing such a structure. The polymer solution 38, which contains the polymer dissolved in a solvent, is contained within the syringe assembly 36. The syringe assembly 36 is part of a syringe pump assembly 32 in which a computer 34 controls the rate at which the polymer solution exits the syringe by controlling pressure or flow rate. Optionally, different flow rates can be provided and controlled to selected spinnerets. The flow rate will change depending on the desired physical characteristics of the polymer scaffold, i.e., membrane thickness, fiber diameter, pore size, membrane density, etc.

The syringe pump assembly 32 feeds the polymer solution to a spinneret 42 that sits on a platform 44. The spinneret has a tip geometry which allows for jet formation and transportation, without interference. A charge in the range of about 10 to about 30 kV is applied to the spinneret by a high voltage power supply 48 through wire 41A.

A mandrel 56A (which, as mentioned in FIG. 3B, includes 55, 57A and 57B) is positioned below the spinneret 42 such that an electric field is created between the charged spinneret and the mandrel 56A. The electric field causes a jet of the polymer solution to be ejected from the spinnerets and spray towards the mandrel 56A, forming micron or nanometer diameter filaments or fibers 46. The drill chucks are grounded using ground wires 41B and 41C.

The mandrel 56A is attached to a first drill chuck 54 (attached to a non-conducting bearing 60) and a second drill chuck 54A (attached to a non-conducting bearing 60A) which is connected to a motor 52. The motor 52 is linked to a speed control 50 which controls the rate at which the motor spins the mandrel 56A. Optionally, different spin rates can be provided. The spin rate will change depending on the desired physical characteristics of the polymer scaffold, i.e., membrane thickness, fiber diameter, pore size, membrane density, etc.

In another exemplary embodiment, the invention provides a seamless conduit for use in a stent produced via the electrospinning apparatus of FIG. 2. This apparatus is similar to the apparatus of FIG. 1 but also comprises a tower 40 which holds the platform 44.

Conduits with multiple layers of polymer scaffolds can be produced in a variety of ways. In an exemplary embodiment, additional polymer scaffold sheets can be wrapped around the outside or the inside of a conduit described herein. In an exemplary embodiment, a longitudinally aligned fibrous polymer scaffold conduit is created, either seamless or with a seam. Then a fibrous polymer scaffold sheet of unaligned micro/nanofibers is placed around the longitudinally aligned conduit to form a two layer conduit with an inner longitudinally aligned fibrous layer and an outer unaligned fibrous layer. Conduits with additional layers (three, four, five, six, etc.) are possible extensions of these methods. Sutures or adhesives can optionally be added to the polymer to maintain this structure.

In another exemplary embodiment, a longitudinally aligned fibrous polymer scaffold conduit is created, either seamless or with a seam. Then a circumferentially aligned fibrous polymer scaffold sheet is placed around the longitudinally aligned conduit to form a two layer conduit with an inner longitudinally aligned fibrous layer and an outer circumferentially aligned fibrous layer. Sutures or adhesives can optionally be added to the seamed polymer scaffold to maintain this structure.

In another exemplary embodiment, a seamless fibrous polymer scaffold conduit is created with an inner wall composed of longitudinally aligned fibers and an outer wall composed of circumferentially aligned fibers. The mandrel described herein with two conducting regions flanking a non-conducting region is used during electrospinning. The mandrel is rotated at a slow speed to allow for the even deposition of longitudinally aligned fibers. The mandrel is then rotated at a high speed to allow for the even deposition of circumferentially aligned fibers. The result is a seamless fibrous polymer scaffold conduit with an inner longitudinally aligned fiber layer and an outer circumferentially aligned fiber layer.

In another exemplary embodiment, a seamless fibrous polymer scaffold conduit is created with an inner wall composed of longitudinally aligned fibers and an outer wall composed of unaligned fibers. The specialized mandrel described above with two conducting regions flanking a non-conducting region is used during electrospinning. The mandrel is rotated at a slow speed to allow for the even deposition of longitudinally aligned fibers. The mandrel is then rotated at an intermediate speed that prevents both longitudinal and circumferential alignment of the deposited fibers. The result is a seamless fibrous polymer scaffold conduit with an inner longitudinally aligned fiber layer and an outer randomly aligned fiber layer.

### Filled Conduit

In an exemplary embodiment, the polymer scaffold has the shape of a filled conduit. The filled conduit can be produced as follows: (1) a conduit is formed as described herein; and (2) filler material for the filled conduit is composed of longitudinally aligned fibers. This filler material can be a loose, highly porous material. In an exemplary embodiment, the filler material is electrospun as a thin membrane of aligned fibers. The material is then directly inserted within the conduit described herein with the orientation of the aligned fibers parallel to the long axis of the conduit. In another instance, a rod of longitudinally aligned fibers is produced as described herein. This rod is then either: (1) directly inserted within a fully formed conduit or (2) used as a mandrel around which a fibrous sheet is rolled and then sealed with sutures or adhesive to form a filled conduit.

### Bifurcated Stent/Graft

Deploying a stent at a bifurcation is challenging because the stent must overlay the entire diseased area of the bifurcation, yet not compromise blood flow. Conventional stents are designed to repair areas of blood vessels that are removed from bifurcations and, since a conventional stent generally terminates at right angles to its longitudinal axis, the use of conventional stents in the region of a vessel bifurcation may result in blocking blood flow of a side branch (commonly referred to as “jailing” the side branch) or fail to repair the bifurcation to the fullest extent necessary. To be effective, the stent preferably overlays the entire circumference of the ostium to a diseased portion and extend to a point within and beyond the diseased portion. Where the stent does not overlay the entire circumference of the ostium to the diseased portion, the stent fails to completely repair the bifurcated vessel. Exemplary formats for stents according to this embodiment are set forth in U.S. Pat. Nos. 6,780,174 and 7,112,217.

To overcome the problems and limitations associated with the use of conventional stents, the present invention provides a Y-shaped stent, Stents according to this format are of use for the treatment of bifurcations. Such a stent has the advantage of completely repairing the vessel at the bifurcation without obstructing blood flow in other portions of the bifurcation. In addition, such a stent allows access to all portions of the bifurcated vessel should further interventional treatment be necessary. In a situation involving disease in the origin of an angulated aorta-ostial vessel, such a stent has the advantage of completely repairing the vessel origin without protruding into the aorta or complicating repeat access.

In various embodiments, the present invention provides a graft system for repairing a region of vascular injury involving a bifurcation of the vasculature. For example, in one embodiment, the invention provides a stent and a method for repairing an aneurysm in an abdominal aorta. An exemplary bifurcated stent of the invention comprises a main body comprising a stent scaffold and a plurality of nanodiameter polymer fibers in contact with at least one face (e.g., inner, outer) of the scaffold. The stent further includes a first side branch coupled to the main body. The first side branch is preferably coupled to the stent scaffold. A second side branch is also coupled to the main body and preferably to the stent scaffold. In an exemplary embodiment, the first side branch the second side branch are coupled to each other at an apex section of the bifurcated stent.

In various embodiments in which the stent is implanted, the bifurcated stent is inserted into a body lumen in a closed configuration with an axis of the first side branch and an axis of the second side branch substantially parallel to each other.

The bifurcated stent can be used with any bifurcated anatomical structure, exemplars of which include the abdominal aorta and branched structures in the lungs.

Other stent architectures will be apparent to those of skill in the art. For example, the stents of the invention can also include a valve, e.g., a one way valve, for inlet and output of blood, emboli, gas, and air. The lengths of exemplary stents of the invention vary from about 1 mm to about 30 mm. For example, a peripheral vascular stent/stent graft of the invention is optionally from about 6 mm to about 15 mm in diameter. A wall stent (carotid or arterial) is from about 5 mm to 10 mm in diameter. An exemplary bronchial/lung segment stent of the invention is from about 5 mm to about 20 mm in length. An exemplary coronary artery stent is from about 2 to about 4 mm in diameter.

### Additional Stent Components

### Cells

A cell can be covalently attached or non-covalently associated with the stents of the invention, the stent and/or the polymer scaffold. Non-covalent association can also be termed “embedded”. In some embodiments, the cell is utilized to promote the growth of new tissue. In an exemplary embodiment, the cell is a member selected from autologous and heterologous. In an exemplary embodiment, the cell is not a stem cell. In an exemplary embodiment, the cell is a stem cell. In an exemplary embodiment, the cell is a member selected from an adult stem cell and an embryonic stem cell. In another exemplary embodiment, the stem cell is a member selected from unipotent, multipotent, pluripotent and totipotent. In an exemplary embodiment, the cell is a progenitor cell. In another exemplary embodiment, the cell is a member selected from adult vascular cells, vascular stem cell or combinations thereof.

Cells can be incorporated within the stents of the invention during electrospinning or post-fabrication. These cells can be incorporated via blending, covalent attachment directly or through various linkers or by adsorption.

### Biomolecules

Vascular restrictions that have been dilated do not always remain open. In up to 50% of the cases, a new restriction in the lumen of the vascular structure appears over a period of months. The newly formed restriction, or “restenosis,” arises due to the onset and maintenance of intimal hyperplasia at the site of insult. Restenosis and intimal hyperplasia following a procedure on a vascular structure is discussed in the following publications, see, for example Khanolkar, Indian Heart J. 48:281 282 (1996); Ghannem et al., Ann. Cardiol. Angeiol. 45:287 290 (1996); Macander et al., Cathet. Cardiovasc. Diagn. 32:125 131; Strauss et al., J. Am. Coll. Cardiol. 20:1465 1473 (1992); Bowerman et al., Cathet. Cardiovasc. Diagn. 24:248 251 (1991); Moris et al., Am. Heart. J. 131:834 836 (1996); Schomig et al., J. Am. Coll. Cardiol. 23:1053 1060 (1994); Gordon et al., J. Am. Coll. Cardiol. 21:1166 1174; and Baim et al., Am. J. Cardiol. 71:364 366 (1993).

Intimal hyperplasia also arises in conjunction with vascular reconstructive surgery. Vascular reconstructive surgery involves removing or reinforcing an area of diseased vasculature. Following removal of the diseased portion of the vessel, a prosthetic device, such as an endovascular stent graft or prosthetic graft is implanted at the site of removal. The graft is typically a segment of autologous or heterologous vasculature or, alternatively, it is a synthetic device fabricated from a polymeric material. Stent grafts are generally fabricated from metals, polymers and combinations of these materials. Similar to the situation with angioplasty, intimal hyperplasia also causes failure of implanted prosthetics in vascular reconstructive surgery. Thus, a method to reduce the failure rate for angioplasty and vascular reconstructive surgery by preventing or reducing intimal hyperplasia is an avidly sought goal.

Intimal hyperplasia is the result of a complex series of biological processes initiated by vascular injury followed by platelet aggregation and thrombus formation with a final pathway of smooth muscle cell migration and proliferation and extracellular matrix deposition. Platelets adhere and aggregate at the site of injury and release biologically active substances, the most important of which are platelet-derived growth factors (Scharf et al., Blut 55:1131 1144 (1987)). It has been postulated that intimal hyperplasia production is driven by two principal mechanisms; platelet activation with the release of platelet-derived growth factors, and activation of the coagulation cascade with thrombus formation, which also results in the release of biologically active substances, which can contribute to smooth muscle cell proliferation (Chervu et al., Surg. Gynecol. Obstet. 171:433 447, 1990)).

Attempts to prevent the onset, or to mitigate the effects, of intimal hyperplasia have included, for example, drug therapy with antihyperplastic agents, such as antiplatelet agents (e.g. aspirin, arachidonic acid, prostacyclin), antibodies to platelet-derived growth factors, and antithrombotic agents (e.g. heparin, low molecular weight heparins) (see, Ragosta et al. Circulation 89: 11262 127 (1994)). Clinical trials using antihyperplastic agents, however, have shown little effect on the rate of restenosis (Schwartz, et al., N. Engl. J. Med. 318:1714 1719, (1988); Meier, Eur. Heart J. 10 (suppl G):64 68 (1989)). In both angioplasty and vascular reconstructive surgery, drug infusion near the site of stenosis has been proposed as a means to inhibit restenosis. For example, U.S. Pat. No. 5,558,642 to Schweich et al. describes drug delivery devices and methods for delivering pharmacological agents to vessel walls in conjunction with angioplasty.

In addition to simply administering a bioactive agent to a patient to prevent restenosis, a number of more sophisticated methods have been investigated. For example, to address the restenosis problem in vascular reconstruction, it has been proposed to provide stents which are seeded with endothelial cells (Dichek et al, Circulation 80:1347 1353 (1989). Both autologous and heterologous cells have been used (see, for example, Williams, U.S. Pat. No. 5,131,907, which issued on Jul. 21, 1992; and Herring, Surgery 84:498 504 (1978)).

Methods of providing therapeutic substances to the vascular wall by means of drug-coated stents have also been proposed. For example, methotrexate and heparin have been incorporated into a cellulose ester stent coating. (Cox et al., Circulation 84: 1171 (1991)). Implanted stents have also been used to carry thrombolytic agents. For example, U.S. Pat. No. 5,163,952 to Froix discloses a thermal memoried expanding plastic stent device, which can be formulated to carry a medicinal agent by utilizing the material of the stent itself as an inert polymeric drug carrier. Pinchuk, in U.S. Pat. No. 5,092,877, discloses a stent of a polymeric material which can be employed with a coating that provides for the delivery of drugs. Ding et al., U.S. Pat. No. 5,837,313 disclose a method of coating an implantable open lattice metallic stent prosthesis with a drug releasing coating.

A biomolecule (such as a pharmaceutical drug, nucleic acid, amino acid, sugar or lipid) can be covalently attached or non-covalently associated with the stents of the invention, the stent and/or the polymer scaffolds described herein. In an exemplary embodiment, the biomolecule is a member selected from a pharmaceutical drug, receptor molecule, extracellular matrix component or a biochemical factor. In another exemplary embodiment, the biochemical factor is a member selected from a growth factor and a differentiation factor. In an exemplary embodiment, the biomolecule is a member selected from glycosaminoglycans and proteoglycans. In an exemplary embodiment, the biomolecule is a member selected from heparin, heparan sulfate, heparan sulfate proteoglycan and combinations thereof.

In various embodiments, the stents of the invention include one or more bioactive agent that is capable of retarding or arresting the formation of intimal hyperplasia is appropriate for incorporation into the fiber of the invention. For reasons of clarity, the discussion that follows is focused on vascular reconstructive surgery involving implanting a vascular graft. Those of skill will readily appreciate that the discussion is generally applicable to other forms of vascular reconstructive surgery, angioplasty and preventing the formation of post-surgical adhesions in other organs and/or internal structures.

Intimal hyperplasia is caused by a cascade of events in response to vascular damage. As part of the inflammatory and reparative response to vascular damage, such as that resulting from vascular surgeries, inflammatory cells (e.g., monocytes, macrophages, and activated polymorphonuclear leukocytes and lymphocytes) often form inflammatory lesions in the blood vessel wall. Lesion formation activates cells in the intimal and medial cellular layers of the blood vessel or heart. The cellular activation may include the migration of cells to the innermost cellular layers, known as the intima. Such migrations pose a problem for the long-term success of vascular grafts because endothelial cells release smooth muscle cell growth factors (e.g., platelet-derived growth factor, interleukin-1, tumor necrosis factor, transforming growth factor-beta, and basic fibroblast growth factor), that cause these newly-migrated smooth muscle cells to proliferate. Additionally, thrombin has been demonstrated to promote smooth muscle cell proliferation both by acting as a growth factor itself and by enhancing the release of several other growth factors produced by platelets and endothelial cells (Wu et al., Annu. Rev. Med. 47:315 31 (1996)). Smooth muscle cell proliferation causes irregular and uncontrolled growth of the intima into the lumen of the blood vessel or heart, which constricts and often closes the vascular passage. Often, irregular calcium deposits in the media or lipid deposits in the intima accompany smooth muscle cell growths, such lipid deposits normally existing in the form of cholesterol and cholesteryl esters that are accumulated within macrophages, T lymphocytes, and smooth muscle cells. These calcium and lipid deposits cause arteriosclerotic hardening of the arteries and veins and eventual vascular failure. These arteriosclerotic lesions caused by vascular grafting can also be removed by additional reconstructive vascular surgery, but the failure rate of this approach due to restenosis has been observed to be between thirty and fifty percent.

Any bioactive agent that can interrupt or retard one or more of the elements of the above-described hyperplastic cascade is useful in practicing the present invention. Example of useful bioactive agents include, but are not limited to, antithrombotics, antiinflammatories, corticosteroids, antimicrotubule agents, antisense oligonucleotides, antineoplastics, antioxidants, antiplatelets, calcium channel blockers, converting enzyme inhibitors, cytokine inhibitors, growth factors, growth factor inhibitors, growth factor sequestering agents, immunosuppressives, tissue factor inhibitor, smooth muscle inhibitors, organoselenium compounds, retinoic acid, retinoid compounds, sulfated proteoglycans, superoxide dismutase mimics, NO, NO precursors and combinations thereof.

Certain biologically active agents falling within the above-recited classes are presently preferred. For example, when one or more of the bioactive agents is an antithrombotic agent, it is preferably selected from heparin, hirudin or a combination thereof. When one or more of the bioactive agents is a corticosteriod, it is preferably selected from dexamethasone, a dexamethosone derivative or a combination thereof. When one or more of the bioactive agents is an antimicrotubule agent, it is preferably selected from taxane, a derivative of taxane or a combination thereof. When one or more of the bioactive agents is an antiplatelet agent, the agent is preferably an inhibitor of collagen synthesis, such as halofuginore, derivatives of halofuginore, proteins (e.g., GpII.sub.bIII.sub.a, ReoPro™) or a combination thereof.

Pharmaceutically acceptable salts of the biologically active agents are also of use in the present invention. Exemplary salts include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

Other agents that are useful in conjunction with the present invention will be readily apparent to those of skill in the art.

The bioactive agents useful in practicing the present invention can be incorporated into the fibers or into coatings on the fibers. One or more of the many art-recognized techniques for immobilizing, coating or adhering, drug molecules to other molecules and surfaces can be used to prepare stents and stent scaffolds incorporating a bioactive agent. These methods include, but are not limited to, covalent attachment to the fibers of the drug or a derivative of the drug bearing a “handle” allowing it to react with a component of the fiber having a complementary reactivity. Moreover, the bioactive agent can be incorporated into the fiber using a non-covalent interaction, such as an electrostatic or an ionic attraction between a charged drug and a component of the fiber bearing a complementary charge. The bioactive agents can also be admixed, and not otherwise interact with, the components of the fiber. The fibers can also be fabricated to incorporate the drugs into reservoirs located in the fibers. The reservoirs can have a variety of shapes, sizes and they can be produced by an array of methods. For example, the reservoir can be a monolithic structure located in one or more components of the fibers. Alternatively, the reservoir can be made up of numerous small microcapsules that are, for example, embedded in the material from which the fiber is fabricated. Furthermore, the reservoir can be a fiber that includes the bioactive agent diffused throughout, or within a portion, of the fiber's three-dimensional structure. The reservoirs can be porous structures that allow the drug to be slowly released from its encapsulation, or the reservoir can include a material that bioerodes following implantation and allows the drug to be released in a controlled fashion.

### Antiplatelet Agents

In an exemplary embodiment, the biomolecule is an antiplatelet agent. Non-limiting examples of antiplatelet agents that may be used in the stents of the invention include adenosine diphosphate (ADP) antagonists or P₂Y₁₂ antagonists, phosphodiesterase (PDE) inhibitors, adenosine reuptake inhibitors, Vitamin K antagonists, heparin, heparin analogs, direct thrombin inhibitors, glycoprotein IIB/IIIA inhibitors, aspirin, non-aspirin NSAIDs, anti-clotting enzymes, as well as pharmaceutically acceptable salts, isomers, enantiomers, polymorphic crystal forms including the amorphous form, solvates, hydrates, co-crystals, complexes, active metabolites, active derivatives and modifications, pro-drugs thereof, and the like.

ADP antagonists or P₂Y₁₂ antagonists block the ADP receptor on platelet cell membranes. This P₂Y₁₂ receptor is important in platelet aggregation, the cross-linking of platelets by fibrin. The blockade of this receptor inhibits platelet aggregation by blocking activation of the glycoprotein IIB/IIIA pathway. In an exemplary embodiment, the antiplatelet agent is an ADP antagonist or P₂Y₁₂ antagonist. In another exemplary embodiment, the antiplatelet agent is a thienopyridine. In another exemplary embodiment, the ADP antagonist or P₂Y₁₂ antagonist is a thienopyridine.

In another exemplary embodiment, the ADP antagonist or P₂Y₁₂ antagonist is a member selected from sulfinpyrazone, ticlopidine, clopidogrel, prasugrel, R-99224 (an active metabolite of prasugrel, supplied by Sankyo), R-1381727, R-125690 (Lilly), C-1330-7, C-50547 (Millennium Pharmaceuticals), INS-48821, INS-48824, INS-446056, INS-46060, INS-49162, INS-49266, INS-50589 (Inspire Pharmaceuticals) and Sch-572423 (Schering Plough). In another exemplary embodiment, the ADP antagonist or P₂Y₁₂ antagonist is ticlopidine hydrochloride (TICLID™). In another exemplary embodiment, the ADP antagonist or P₂Y₁₂ antagonist is a member selected from sulfinpyrazone, ticlopidine, AZD6140, clopidogrel, prasugrel and mixtures thereof. In another exemplary embodiment, the ADP antagonist or P₂Y₁₂ antagonist is clopidogrel. In another exemplary embodiment, the ADP antagonist or P₂Y₁₂ antagonist is a member selected from clopidogrel bisulfate (PLAVIX™), clopidogrel hydrogen sulphate, clopidogrel hydrobromide, clopidogrel mesylate, cangrelor tetrasodium (AR-09931 MX), ARL67085, AR-C66096 AR-C 126532, and AZD-6140 (AstraZeneca). In another exemplary embodiment, the ADP antagonist or P₂Y₁₂ antagonist is prasugrel. In another exemplary embodiment, the ADP antagonist or P₂Y₁₂ antagonist is a member selected from clopidogrel, ticlopidine, sulfinapyrazone, AZD6140, prasugrel and mixtures thereof.

A PDE inhibitor is a drug that blocks one or more of the five subtypes of the enzyme phosphodiesterase (PDE), preventing the inactivation of the intracellular second messengers, cyclic adenosine monophosphate (cAMP) and cyclic guanosine monophosphate (cGMP), by the respective PDE subtype(s). In an exemplary embodiment, the antiplatelet agent is a PDE inhibitor. In an exemplary embodiment, the antiplatelet agent is a selective cAMP PDE inhibitor. In an exemplary embodiment, the PDE inhibitor is cilostazol (Pletal™).

Adenosine reuptake inhibitors prevent the cellular reuptake of adenosine into platelets, red blood cells and endothelial cells, leading to increased extracellular concentrations of adenosine. These compounds inhibit platelet aggregation and cause vasodilation. In an exemplary embodiment, the antiplatelet agent is an adenosine reuptake inhibitor. In an exemplary embodiment, the adenosine reuptake inhibitor is dipyridamole (Persantine™).

Vitamin K inhibitors are given to people to stop thrombosis (blood clotting inappropriately in the blood vessels). This is useful in primary and secondary prevention of deep vein thrombosis, pulmonary embolism, myocardial infarctions and strokes in those who are predisposed. In an exemplary embodiment, the anti-platelet agent is a Vitamin K inhibitor. In an exemplary embodiment, the Vitamin K inhibitor is a member selected from acenocoumarol, clorindione, dicumarol (Dicoumarol), diphenadione, ethyl biscoumacetate, phenprocoumon, phenindione, tioclomarol and warfarin.

Heparin is a biological substance, sometimes made from pig intestines. It works by activating antithrombin III, which blocks thrombin from clotting blood. In an exemplary embodiment, the antiplatelet agent is heparin or a prodrug of heparin. In an exemplary embodiment, the antiplatelet agent is a heparin analog or a prodrug of a heparin analog. In an exemplary embodiment, the heparin analog a member selected from Antithrombin III, Bemiparin, Dalteparin, Danaparoid, Enoxaparin, Fondaparinux (subcutaneous), Nadroparin, Parnaparin, Reviparin, Sulodexide, and Tinzaparin.

Direct thrombin inhibitors (DTIs) are a class of medication that act as anticoagulants (delaying blood clotting) by directly inhibiting the enzyme thrombin. In an exemplary embodiment, the antiplatelet agent is a DTI. In another exemplary embodiment, the DTI is univalent. In another exemplary embodiment, the DTI is bivalent. In an exemplary embodiment, the DTI is a member selected from hirudin, bivalirudin (IV), lepirudin, desirudin, argatroban (IV), dabigatran, dabigatran etexilate (oral formulation), melagatran, ximelagatran (oral formulation but liver complications) and prodrugs thereof.

Glycoprotein IIB/IIIA inhibitors work by inhibiting the GpIIb/IIIa receptor on the surface of platelets, thus preventing platelet aggregation and thrombus formation. In an exemplary embodiment, the antiplatelet agent is a glycoprotein IIB/IIIA inhibitor. In an exemplary embodiment, the glycoprotein IIB/IIIA inhibitor is a member selected from abciximab, eptifibatide, tirofiban and prodrugs thereof. Since these drugs are only administered intravenously, a prodrug of a glycoprotein IIB/IIIA inhibitor is useful for oral administration.

Anti-clotting enzymes may also be used in the invention. In an exemplary embodiment, the antiplatelet agent is an anti-clotting enzyme which is in a form suitable for oral administration. In another exemplary embodiment, the anti-clotting enzyme is a member selected from Alteplase, Ancrod, Anistreplase, Brinase, Drotrecogin alfa, Fibrinolysin, Protein C, Reteplase, Saruplase, Streptokinase, Tenecteplase, Urokinase.

In an exemplary embodiment, the anti-platelet agent is a member selected from aloxiprin, beraprost, carbasalate calcium, cloricromen, defibrotide, ditazole, epoprostenol, indobufen, iloprost, picotamide, rivaroxaban (oral FXa inhibitor) treprostinil, triflusal, or prodrugs thereof.

Methods of Attachina a Biomolecule and/or a Cell to a Biomimetic Scaffold

The biomolecules and/or cell can be attached to the biomimetic scaffold in a variety of ways. In one embodiment, the biomolecule can be non-covalently embedded or absorbed into a first fibrous polymer scaffold described herein.

In another exemplary embodiment, the biomolecule is covalently attached, either directly or through a linker, to a first fibrous polymer scaffold. This covalent attachment is made from the reaction of complementary reactive groups on the first fibrous scaffold and the biomolecule or cell, or between the linker on the first fibrous scaffold and the biomolecule or cell, or between the first fibrous scaffold and the linker on the biomolecule or cell.

### Covalently Attached Bioactive Materials

In a preferred embodiment, the biologically active material is covalently bonded to a reactive group located on one or more components of the fiber. The art is replete with methods for preparing derivatized, polymerizable monomers, attaching bioactive materials onto polymeric surfaces and derivatizing bioactive materials and polymers to allow for this attachment (see, for example, Hermanson, BIOCONJUGATE TECHNIQUES, Academic Press, 1996, and references therein). Common approaches include the use of coupling agents such as glutaraldehyde, cyanogen bromide, p-benzoquinone, succinic anhydrides, carbodiimides, diisocyanates, ethyl chloroformate, dipyridyl disulfide, epichlorohydrin, azides, among others, which serve as attachment vehicles for coupling reactive groups of biologically active molecules to reactive groups on a monomer or a polymer.

Complementary reactive functional groups and classes of reactions useful in practicing the present invention are generally those that are well known in the art of bioconjugate chemistry. Currently favored classes of reactions available with reactive functional groups of the invention are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

In an exemplary embodiment, the reactive functional groups are members selected from

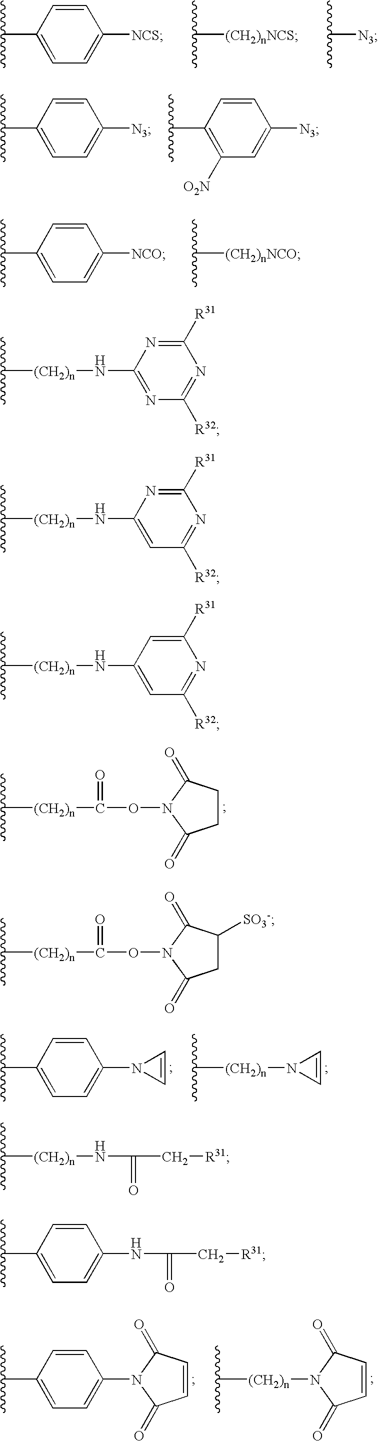

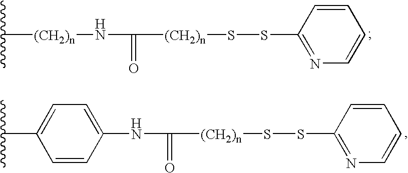

wherein R³¹ and R³² are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

Additional examples of reactive functional groups, as well as the corresponding functional groups with which they react, are provided in the following table:

TABLE 1
Reactive Substituents and Sites Reactive Therewith

| Reactive Functional Groups | Corresponding Functional Groups |
|---|---|
| Succinimidyl esters | primary amino, secondary amino, hydroxyl |
| Anhydrides | primary amino, secondary amino, hydroxyl |
| Acyl azides | primary amino, secondary amino |
| Isothiocyanates, isocyanates | amino, thiol, hydroxyl |
| sulfonyl chlorides | amino, hydroxyl |
| sulfonyl fluorides | |
| hydrazines, | aldehydes, ketones |
| substituted hydrazines | |
| hydroxylamines, | amino, hydroxyl |
| substituted hydroxylamines | |
| acid halides | amino, hydroxyl |
| haloacetamides, maleimides | thiol, imidazoles, hydroxyl, amino |
| carbodiimides | carboxyl groups |
| phosphoramidites | hydroxyl |
| azides | alkynes |

For example, in order to attach a compound of the invention to the hydroxyl moiety on a serine amino acid, exemplary reactive functional groups include, succinimidyl esters, anhydrides, isothiocyanates, thiocyanates, sulfonyl chlorides, sulfonyl fluorides, acid halides, haloacetamides, maleimides and phosphoramidites.

The reactive functional groups can be chosen such that they do not participate in, or interfere with, the reactions necessary to assemble the compound of the invention. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

In an exemplary embodiment, a carboxylic acid moiety is being attached to an amine moiety. First the carboxylic acid moiety is reacted with a carbodiimide to generate an O-acylisourea, which can react with the amine moiety to produce an amide which then links the two moieties.

In an exemplary embodiment, attachment of biomolecules to a fibrous polymer scaffold described herein can be achieved by any of several methods. For example, a biomolecule (such as an antiplatelet agent) can be attached to a polymer scaffold described herein optionally containing a linking group by treatment with EDC/sulfo-NHS (i.e., 1-ethyl-3(3-dimethylaminopropylcarbodiimide/N-hydroxysulfo-succinimide), which will facilitate linkage of, for example, the amino end of the fibrous polymer to the carboxyl group of the biomolecule, or to an amine-containing linker bound to the fibrous polymer. Please note that the linkages could be on the opposite species as well (the carboxyl group could be on the fibrous polymer and the amine group could be on the biomolecule). As will be readily recognized by one of ordinary skill in the art EDC/sulfo-NHS can be replaced by any suitable group including, but not limited to, N-5-azido-2-nitrobenzoyloxysuccinimide; p-azidophenacyl bromide; p-azidophenyl glyoxal; n-4-(azidophenylthio)phthalimide; bis(sulfosuccinimidyl)suberate; bis maleimidohexane; bis[2-(succinimidooxycarbonyloxy)-ethyl]sulfone; 1,5-difluoro-2,4-dinitrobenzene; 4,4′-diisothiocyano-2,2′-disulfonic acid stilbene; dimethyl adipimidate-2HCl; dimethyl pimelimidate-2HCl; dimethyl suberimidate-2HCl; dithiobis(succinimidylpropionate); disuccinimidyl suberate; disuccinimidyl tartarate; dimethyl 3,3′-dithiobispropionimidate-2-HCl; 4,4′-diothiobisphenylazide; 3,3-dithiobis(sulfosuccinimidyl-propionate); ethyl-4-azidophenyl 1,4-dithio-butyrimidate; ethylene glycolbis(succinimidyl-succinate); 1-azido-4-fluoro-3-nitobenzene; N-hydroxysuccinimidyl-4-azidobenzoate; methyl-4-azidobenzoimidate; m-maleimidobenzoyl-N-hydroxysulfo-succinimide ester; N-hydroxysuccinimidyl-4-azidosalicylic acid; p-nitrophenyl-2-diazo-3,3,3-trifluoro-propionate; N-succinimidyl(4-axidophenyl)-1,3′-di-thiopropionate; sulfosuccinimidyl 2-(m-azido-o-nitro-benzamido)-ethyl-1,3′-dithiopropionate; N-succinimidyl-6(4′-azido-2′-nitro-phenyl-amino)hexanoate; sulfosuccinimidyl 2-(p-azidosalicyl-amido)ethyl-1,3′-dithio-propionate; N-succinimidyl(4-iodoacetyl)amino-benzoate; succinimidyl 4-(N-maleimidomethyl)-cyclohexane-1-carboxylate; succinimidyl 4-(p-maleimidophenyl)-butyrate; N-succinimidyl 3-(2-pyridyldithio)propionate; bis[2-(sulfosuccinimidooxy-carbonyl-oxy)ethyl]sulfone; disulfosuccinimidyl tartarate; ethylene glycolbis(sulfosuccinimidyl-succinate; m-maleimidobenzoyl-N-hydroxysulfo-succinimide ester; sulfosuccinimidyl(4-azidophenyldithio)-propionate; sulfosuccinimidyl 6-(4′azido-2′-nithro-phenylamino)hexanoate-sulfosuccinimidyl(4-iodoacetyl)amino-benzoate; sulfosuccinimidyl 4-(N-maleimido-methyl)cyclohexane-1-carboxylate; sulfosuccinimidyl 4-(p-maleimido-phenyl)butyrate; and 2-iminothiolane-HCl.

A polymer can be functionalized with reactive groups by, for example, including a moiety bearing a reactive group as an additive to a blend during manufacture of the polymer or polymer precursor. The additive is dispersed throughout the polymer matrix, but does not form an integral part of the polymeric backbone. In this embodiment, the surface of the polymeric material is altered or manipulated by the choice of additive or modifier characteristics. The reactive groups of the additive are used to bind one or more bioactive agents to the polymer.

A useful method of preparing surface-functionalized polymeric materials by this method is set forth in, for example, Caldwell, U.S. Pat. No. 5,874,164, issued Feb. 23, 1999. In the Caldwell method, additives or modifiers are combined with the polymeric material during its manufacture. These additives or modifiers include compounds that have reactive sites, compounds that facilitate the controlled release of agents from the polymeric material into the surrounding environment, catalysts, compounds that promote adhesion between the bioactive materials and the polymeric material and compounds that alter the surface chemistry of the polymeric material.

In another embodiment, polymerizable monomers bearing reactive groups are incorporated in the polymerization mixture. The functionalized monomers form part of the polymeric backbone and, preferably, present their reactive groups on the surface of the polymer.

Reactive groups contemplated in the practice of the present invention include functional groups, such as hydroxyl, carboxyl, carboxylic acid, amine groups, and the like, that promote physical and/or chemical interaction with the bioactive material. The particular compound employed as the modifier will depend on the chemical functionality of the biologically active agent and can readily be deduced by one of skill in the art. In the present embodiment, the reactive site binds a bioactive agent by covalent means. It will, however, be apparent to those of skill in the art that these reactive groups can also be used to adhere bioactive agents to the polymer by hydrophobic/hydrophilic, ionic and other non-covalent mechanisms.

In addition to manipulating the composition and structure of the polymer during manufacture, a preferred polymer can also be modified using a surface derivitization technique. There are a number of surface-derivatization techniques appropriate for use in fabricating the delivery vehicles of the present invention (e.g., grafting techniques). These techniques for creating functionalized polymeric surfaces are well known to those skilled in the art. For example, techniques based on ceric ion initiation, ozone exposure, corona discharge, UV irradiation and ionizing radiation (.sup.60Co, X-rays, high energy electrons, plasma gas discharge) are known and can be used in the practice of the present invention.

Substantially any reactive group that can be reacted with a complementary component on a biologically active material can be incorporated into a polymer and used to covalently attach the biologically active material to the fiber of use in the invention. In a preferred embodiment, the reactive group is selected from amine-containing groups, hydroxyl groups, carboxyl groups, carbonyl groups, and combinations thereof. In a further preferred embodiment, the reactive group is an amino group.

Synthesis of specific biologically active material-polymer conjugates is generally accomplished by: 1) providing a fiber component comprising an activated polymer, such as an acrylic acid, and a biologically active agent having a position thereon which will allow a linkage to form; 2) reacting the complementary substituents of the biologically active agent and the fiber component in an inert solvent, such as methylene chloride, chloroform or DMF, in the presence of a coupling reagent, such as 1,3-diisopropylcarbodiimide or any suitable dialkyl carbodiimide (Sigma Chemical), and a base, such as dimethylaminopyridine, diisopropyl ethylamine, pyridine, triethylamine, etc. Alternative specific syntheses are readily accessible to those of skill in the art (see, for example, Greenwald et al., U.S. Pat. No. 5,880,131, issued Mar. 9, 1999).

By way of example, the discussion below is concerned with the attachment of a peptide-based bioactive material to an amine-containing polymeric component of a fiber of use in practicing the methods of the invention. The choice of a peptide-based biologically active material and an amine-containing polymer is intended to be illustrative of the invention and does not define its scope. It will be apparent to those of skill in the art how to attach a wide range of biologically active agents to polymers comprising amines and other reactive groups.

The conjugates of use in practicing the instant invention, which comprise a peptide, can be synthesized by techniques well known in the medicinal chemistry art. For example, a free amine moiety on a polymeric fiber component can be covalently attached to an oligopeptide at the carboxyl terminus such that an amide bond is formed. Similarly, an amide bond may be formed by covalently coupling an amine moiety of an oligopeptide and a carboxyl moiety of a polymeric fiber component. For these purposes, a reagent such as 2-(1H-benzotriazol-1-yl)-1,3,3-tetramethyluronium hexafluorophosphate (known as HBTU) and 1-hyroxybenzotriazole hydrate (known as HOBT), dicyclohexylcarbodiimide (DCC), N-ethyl-N-(3-dimethylaminopropyl)-carbodiimide (EDC), diphenylphosphorylazide (DPPA), benzotriazol-1-yl-oxy-tris-(dimethylamino)phosphonium hexafluorophosphate (BOP) and the like, in combination, or singularly, can be utilized.

Furthermore, the instant conjugate can be formed by a non-peptidyl bond between a peptide and a fiber component. For example, a peptide can be attached to a fiber component through a carboxyl terminus of an oligopeptide via a hydroxyl moiety on a polymeric fiber component, thereby forming an ester linkage. For this purpose, a reagent such as a combination of HBTU and HOBT, a combination of BOP and imidazole, a combination of DCC and DMAP, and the like can be utilized.

The instant conjugate can also be formed by attaching the oligopeptide to the polymeric fiber component using a linker unit. Such linker units include, for example, a biscarbonyl alkyl diradical whereby an amine moiety on the fiber component is connected with the linker unit to form an amide bond and the amino terminus of the oligopeptide is connected with the other end of the linker unit also forming an amide bond. Conversely, a diaminoalkyl diradical linker unit, whereby a carbonyl moiety on the fiber component is covalently attached to one of the amines of the linker unit while the other amine of the linker unit is covalently attached to the C-terminus of the oligopeptide, can also be utilized. Other such linker units, which are stable to the physiological environment, are also envisioned.

In addition to linkers that are stable in vivo, linkers that are designed to be cleaved to release the biologically active agent from the polymer are useful in the methods of the present invention. Many such linker arms are accessible to those of skill in the art. Common cleavable linker arms include, for example, specific protease cleavage sequences, disulfides, esters and the like. Many appropriate cleavable cross-linking agents are commercially available from companies, such as Pierce (Rockford, Ill.), or can be prepared by art-recognized methods.

Any of the bioactive agents from the various classes of bioactive agents set forth above can be tethered to a polymer by the methods described herein. In a particularly preferred embodiment, the biologically active material is a taxane. For purposes of the present invention, the term “taxane” includes all compounds within the taxane family of terpenes. Thus, taxol (paclitaxel), 3′-substituted tert-butoxy-carbonyl-amine derivatives (taxoteres) and the like as well as other analogs available from, for example, Sigma Chemical (St. Louis, Mo.) and/or Bristol Meyers Squibb are within the scope of the present invention.

Generally, it is preferred that a taxane having the 2′ position available for substitution is reacted with a suitably activated polymer such as a polymeric carboxylic acid under conditions sufficient to cause the formation of a 2′ ester linkage between the two substituents.

One skilled in the art understands that in the synthesis of compounds useful in practicing the present invention, one may need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to PROTECTIVE GROUPS IN ORGANIC CHEMISTRY, McOmie, ed., Plenum Press, NY, N.Y. (1973); and, PROTECTIVE GROUPS IN ORGANIC SYNTHESIS, Greene, ed., John Wiley & Sons, NY (1981) for the teaching of protective groups which may be useful in the preparation of compounds of the present invention.

In another exemplary embodiment, the biomolecule or antiplatelet agent is covalently attached to the first fibrous polymer and/or the stent without a linker. In another exemplary embodiment, the invention provides a stent including a structure which is a member selected from the following formulae:

A-X-Biomolecule (I) or

A-X-Antiplateletagent (Ia)

wherein A is a first fibrous polymer scaffold, X is a member selected from a covalent bond, O, S, C(O), C(O)O, C(O)S, C(O)NH, S(O), S(O)₂, S(O)₂NR*,C(O)NR*, NH or NR* wherein R* is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In an exemplary embodiment, the antiplatelet agent is a member selected from heparin, hirudin, bivalirudin, lepirudin, desirudin, argatroban, dabigatran, dabigatran etexilate, melagatran, ximelagatran and prodrugs thereof. In an exemplary embodiment, the antiplatelet agent is a member selected from hirudin, hirudin analogs, bivalirudin, lepirudin and desirudin. In another exemplary embodiment, the antiplatelet agent comprises a thrombin binding site. In another exemplary embodiment, the antiplatelet agent comprises a protein sequence which is a member selected from FPRP and LYEEPIEEFDGN. In another exemplary embodiment, the first fibrous polymer scaffold is seamless. In another exemplary embodiment, the first fibrous polymer scaffold is monolithically formed.

In an exemplary embodiment, the stent comprises at least one moiety having a structure which is a member according to the following formulae:

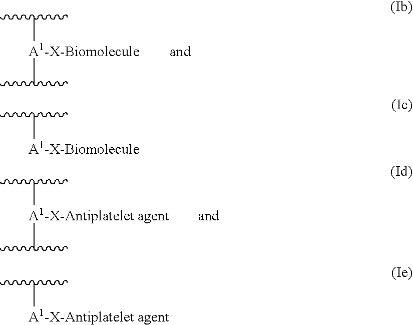

wherein the symbol indicates the point at which the displayed moiety is attached to the remainder of the polymer, A¹ is a subunit of the first fibrous polymer scaffold. In an exemplary embodiment, A¹ is a subunit which is formed by polymerization of a monomer/series of monomers. In an exemplary embodiment, A¹ is a member selected from one or more of the monomers or subunits described herein. In another exemplary embodiment, A¹ is a member selected from an aliphatic polyester, a polyalkylene oxide, polydimethylsiloxane, polyvinylalcohol, polylysine, collagen, laminin, fibronectin, elastin, alginate, fibrin, hyaluronic acid, proteoglycans, polypeptides and combinations thereof. In another exemplary embodiment, A¹ is a functionalized lactide moiety. In an exemplary embodiment, said functionalized lactide moiety is functionalized at the side chain methyl. In another exemplary embodiment, X is derived from the reaction of complementary reactive groups on the first fibrous scaffold and the antiplatelet agent. Descriptions of these complementary reactive groups are provided herein. In an exemplary embodiment, the antiplatelet agent is a member selected from hirudin, hirudin analogs, bivalirudin, lepirudin and desirudin. In another exemplary embodiment, the antiplatelet agent comprises a thrombin binding site. In another exemplary embodiment, the antiplatelet agent comprises a protein sequence which is a member selected from FPRP and LYEEPIEEFDGN. In another exemplary embodiment, the first fibrous polymer scaffold is seamless. In another exemplary embodiment, the first fibrous polymer scaffold is monolithically formed.

In an exemplary embodiment, the stent comprises at least one moiety having a structure which is a member selected from the following formulae:

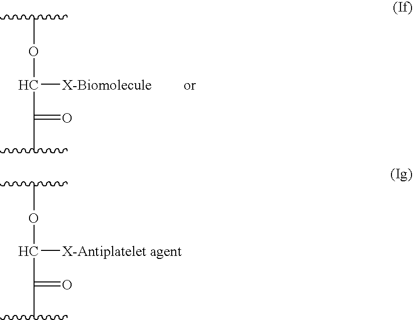

wherein the symbol indicates the point at which the displayed moiety is attached to the remainder of the polymer, X is a member selected from a covalent bond, O, S, C(O), C(O)O, C(O)S, C(O)NH, S(O), S(O)₂, S(O)₂NR*, C(O)NR*, NH or NR* wherein R* is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In an exemplary embodiment, X is a member selected from C(O)NH and C(O)NR*. In an exemplary embodiment, the antiplatelet agent is a member selected from heparin, hirudin, bivalirudin, lepirudin, desirudin, argatroban, dabigatran, dabigatran etexilate, melagatran, ximelagatran and prodrugs thereof. In an exemplary embodiment, the antiplatelet agent is a member selected from hirudin, hirudin analogs, bivalirudin, lepirudin and desirudin. In another exemplary embodiment, the antiplatelet agent comprises a thrombin binding site. In another exemplary embodiment, the antiplatelet agent comprises a protein sequence which is a member selected from FPRP and LYEEPIEEFDGN. In another exemplary embodiment, X is attached to the C-terminal domain of hirudin. In an exemplary embodiment, X is attached to the amino acid at the C-terminus of hirudin. In an exemplary embodiment, X is attached to hirudin through the side-chain of an aspartic acid or glutamic acid moiety. In an exemplary embodiment, X is attached to hirudin through the side-chain of an aspartic acid moiety. In an exemplary embodiment, X is attached to an amino acid moiety of hirudin which is a member selected from D5, E8, E17, D33, E35, E43, D53, D55, E57, E58, E61, E62 and Q65 of the wild-type peptide sequence. In another exemplary embodiment, the first fibrous polymer scaffold is seamless. In another exemplary embodiment, the first fibrous polymer scaffold is monolithically formed.

In another exemplary embodiment, the invention provides a stent having a structure which is a member selected from the following formulae:

A-X-L-Biomolecule (II)

A-X-L-Antiplateletagent (IIa)

wherein A is a first fibrous polymer scaffold, X is a member selected from a covalent bond, O, S, C(O), C(O)O, C(O)S, C(O)NH, S(O), S(O)₂, S(O)₂NR*, C(O)NR*, NH or NR* wherein R* is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, L is a linker, and the antiplatelet agent is a member including, but not limited to, heparin, hirudin, bivalirudin, lepirudin, desirudin, argatroban, dabigatran, dabigatran etexilate (oral formulation), melagatran, ximelagatran and prodrugs thereof. In an exemplary embodiment, A is a first fibrous polymer scaffold conduit or filled conduit, wherein the fiber or fibers of the first fibrous polymer scaffold are aligned. In an exemplary embodiment, L includes a member selected from polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly(amino acids), e.g., poly(aspartic acid) and poly(glutamic acid); nucleic acids and copolymers thereof. In another exemplary embodiment, L includes a member selected from a peptide, saccharide, poly(ether), poly(amine), poly(carboxylic acid), poly(alkylene glycol), such as poly(ethylene glycol) (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), polysialic acid, polyglutamate, polyaspartate, polylysine, polyethyeleneimine, biodegradable polymers (e.g., polylactide, polyglyceride and copolymers thereof), polyacrylic acid. In an exemplary embodiment, L is a member selected from polyethylene glycol and polypropylene glycol. In another exemplary embodiment, X is N and L is a member selected from polyethylene glycol and polypropylene glycol. In an exemplary embodiment, the antiplatelet agent is a member selected from hirudin, hirudin analogs, bivalirudin, lepirudin and desirudin. In an exemplary embodiment, A is a first fibrous polymer scaffold conduit or filled conduit. The fiber or fibers of the first fibrous polymer scaffold are aligned. In another exemplary embodiment, the first fibrous polymer scaffold is seamless. In another exemplary embodiment, the first fibrous polymer scaffold is monolithically formed.

In another exemplary embodiment, the biomolecule or antiplatelet agent is covalently attached to the first fibrous polymer and/or the stent with a linker. In an exemplary embodiment, the stent comprises at least one moiety having a structure according to the following formulae:

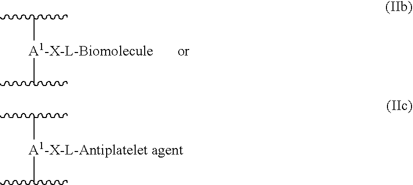

wherein A¹ is as described herein. In another exemplary embodiment, the at least one moiety has a structure according to the following formula:

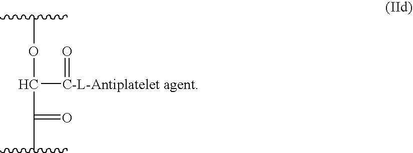

In another exemplary embodiment, the at least one moiety has a structure according to the following formula:

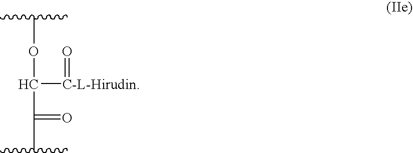

In another exemplary embodiment, hirudin is a member selected from wild-type hirudin, hirudin analogs, bivalirudin, lepirudin and desirudin. In another exemplary embodiment, the antiplatelet agent comprises a thrombin binding site. In another exemplary embodiment, the antiplatelet agent comprises a protein sequence which is a member selected from FPRP and LYEEPIEEFDGN. In another exemplary embodiment, the first fibrous polymer scaffold is seamless. In another exemplary embodiment, the first fibrous polymer scaffold is monolithically formed.

In another exemplary embodiment, the at least one moiety has a structure according to the following formula:

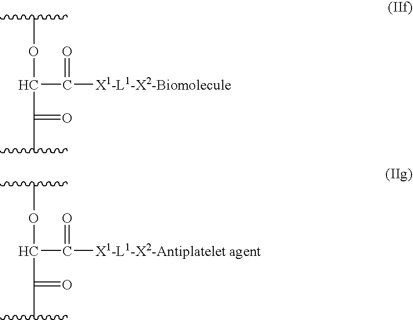

wherein X¹ and X² are members independently selected from a covalent bond, O, S, NH or NR*, wherein R* is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, L¹ is a member selected from a water soluble and water insoluble polymer. In an exemplary embodiment, L¹ includes a member selected from polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly(amino acids), e.g., poly(aspartic acid) and poly(glutamic acid); nucleic acids and copolymers thereof. In another exemplary embodiment, L¹ includes a member selected from a peptide, saccharide, poly(ether), poly(amine), poly(carboxylic acid), poly(alkylene glycol), such as poly(ethylene glycol) (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), polysialic acid, polyglutamate, polyaspartate, polylysine, polyethyeleneimine, biodegradable polymers (e.g., polylactide, polyglyceride and copolymers thereof), polyacrylic acid. In an exemplary embodiment, L¹ is a member selected from PEG, PPG and copolymers thereof. In an exemplary embodiment, L¹ is PEG, wherein said PEG comprises a number of monomeric subunits which is an integer from 1 to 5000. In an exemplary embodiment, said number of monomeric subunits is an integer from about 10 to about 1000. In an exemplary embodiment, said number of monomeric subunits is an integer from about 10 to about 500. In an exemplary embodiment, said number of monomeric subunits is an integer from about 20 to about 400. In an exemplary embodiment, said number of monomeric subunits is an integer from about 20 to about 250. In an exemplary embodiment, said number of monomeric subunits is an integer from about 50 to about 200. In an exemplary embodiment, said number of monomeric subunits is an integer from about 50 to about 125. In an exemplary embodiment, said number of monomeric subunits is an integer from about 50 to about 100. In an exemplary embodiment, said number of monomeric subunits is an integer from about 60 to about 90. In an exemplary embodiment, said number of monomeric subunits is an integer from about 60 to about 90. In another exemplary embodiment, the first fibrous polymer scaffold is seamless. In another exemplary embodiment, the first fibrous polymer scaffold is monolithically formed.

In another exemplary embodiment, the at least one moiety has a structure according to the following formula:

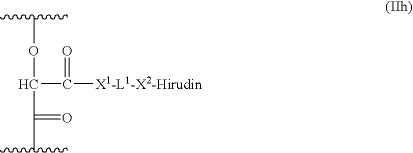

wherein X¹, L¹ and X² are defined as described herein. In an exemplary embodiment, X² is attached to the C-terminal domain of hirudin. In an exemplary embodiment, X² is attached to the amino acid at the C-terminus of hirudin. In an exemplary embodiment, X² is attached to hirudin through the side-chain of an aspartic acid or glutamic acid moiety. In an exemplary embodiment, X² is attached to hirudin through the side-chain of an aspartic acid or glutamic acid moiety. In an exemplary embodiment, X² is attached to an amino acid moiety of hirudin which is a member selected from D5, E8, E17, D33, E35, E43, D53, D55, E57, E58, E61, E62 and Q65. In another exemplary embodiment, the antiplatelet agent comprises a thrombin binding site. In another exemplary embodiment, the antiplatelet agent comprises a protein sequence which is a member selected from FPRP and LYEEPIEEFDGN. In another exemplary embodiment, the first fibrous polymer scaffold is seamless. In another exemplary embodiment, the first fibrous polymer scaffold is monolithically formed.

In another exemplary embodiment, the at least one moiety has a structure according to the following formula:

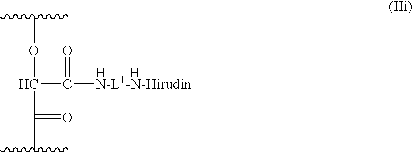

wherein X¹, L¹ and X² are defined as described herein. In an exemplary embodiment, L¹ is PEG. In an exemplary embodiment, hirudin is attached through its C-terminal domain. In an exemplary embodiment, X² is attached to the amino acid at the C-terminus of hirudin. In an exemplary embodiment, X² is attached to hirudin through the side-chain of an aspartic acid or glutamic acid moiety. In an exemplary embodiment, X² is attached to hirudin through the side-chain of an aspartic acid or glutamic acid moiety. In an exemplary embodiment, X² is attached to an amino acid moiety of hirudin which is a member selected from D5, E8, E17, D33, E35, E43, D53, D55, E57, E58, E61, E62 and Q65. In another exemplary embodiment, the antiplatelet agent comprises a thrombin binding site. In another exemplary embodiment, the antiplatelet agent comprises a protein sequence which is a member selected from FPRP and LYEEPIEEFDGN.

In another exemplary embodiment, the invention provides a stent comprising the following structure of Formula III:

A-X¹-L¹-X²-L²-Antiplatelet agent Formula III

wherein A is a first fibrous polymer scaffold, X¹ is an optional first member selected from a covalent bond, O, S, NH or NR, L¹ is an optional first linker, X² is an optional second member selected from a covalent bond, O, S, NH or NR, L² is an optional second linker, and the antiplatelet agent is a member including, but not limited to, heparin, hirudin, bivalirudin, lepirudin, desirudin, argatroban, dabigatran, dabigatran etexilate (oral formulation), melagatran, ximelagatran and prodrugs thereof.

In an exemplary embodiment, A is a first fibrous polymer scaffold conduit or filled conduit, wherein the fiber or fibers of the first fibrous polymer scaffold are aligned. In another exemplary embodiment, each of L¹ and L² is independently a member selected from polyethylene glycol and polypropylene glycol. In another exemplary embodiment, each X is N and L¹ and L² are each a member selected from polyethylene glycol and polypropylene glycol. In an exemplary embodiment, the antiplatelet agent is a member selected from hirudin, hirudin analogs, bivalirudin, lepirudin and desirudin.

In another exemplary embodiment, the invention provides stents comprising the following structure of Formula IV:

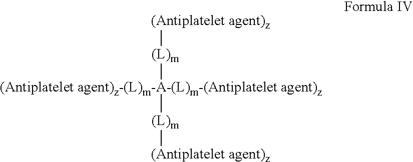

wherein A is a first fibrous polymer scaffold, each L is independently a first linker, which is independently selected from polyethylene glycol and polypropylene glycol, wherein m is an integer from about 0 to about 100, from about 1 to about 50, from about 1 to about 10; and each antiplatelet agent is independently selected from heparin, hirudin, bivalirudin, lepirudin, desirudin, argatroban, dabigatran, dabigatran etexilate (oral formulation), melagatran, ximelagatran and prodrugs thereof, wherein z is an integer from 0 to 1.

In an exemplary embodiment, the antiplatelet agent is a member selected from hirudin, hirudin analogs, bivalirudin, lepirudin and desirudin, wherein z is 1. In another exemplary embodiment, each L is a member selected from polyethylene glycol and polypropylene glycol. In an exemplary embodiment, A is a first fibrous polymer scaffold conduit or filled conduit, wherein the fiber or fibers of the first fibrous polymer scaffold are aligned. In an exemplary embodiment, the antiplatelet agent is a member selected from hirudin, hirudin analogs, bivalirudin, lepirudin and desirudin.

In another exemplary embodiment, the invention provides stents comprising the following structure of Formula V:

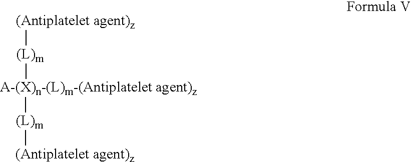

wherein A is a first fibrous polymer scaffold, each X is independently a first member selected from a covalent bond, O, S, NH or NR, wherein n is an integer from about 0 to about 100, from about 1 to about 50, from about 1-10; each L is independently a first linker, which is independently selected from polyethylene glycol and polypropylene glycol, wherein m is an integer from about 0 to about 100, from about 1 to about 50, from about 1 to about 10; and each antiplatelet agent is independently selected from heparin, hirudin, bivalirudin, lepirudin, desirudin, argatroban, dabigatran, dabigatran etexilate (oral formulation), melagatran, ximelagatran and prodrugs thereof. In an exemplary embodiment, the antiplatelet agent is a member selected from hirudin, hirudin analogs, bivalirudin, lepirudin and desirudin, wherein z is an integer from 0 to 1. In another exemplary embodiment, X is N and each L is a member selected from polyethylene glycol and polypropylene glycol. In an exemplary embodiment, A is a first fibrous polymer scaffold conduit or filled conduit, wherein the fiber or fibers of the first fibrous polymer scaffold are aligned. In an exemplary embodiment, each antiplatelet agent is independently a member selected from hirudin, hirudin analogs, bivalirudin, lepirudin and desirudin.

In another exemplary embodiment, the invention provides a stent comprising the following structure of Formula VI:

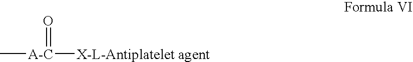

wherein A is a first fibrous polymer scaffold, X is a member selected from O, S, NH or NR, and L is a linker. In an exemplary embodiment, A is a first fibrous polymer scaffold conduit or filled conduit. The fiber or fibers of the first fibrous polymer scaffold are aligned. In an exemplary embodiment, L is a member selected from polyethylene glycol and polypropylene glycol. In another exemplary embodiment, X is N and L is a member selected from polyethylene glycol and polypropylene glycol. In another exemplary embodiment, the C═O moiety is part of a lactide moiety in the first fibrous polymer scaffold. In another exemplary embodiment, the C═O moiety is part of a glycolide moiety in the first fibrous polymer scaffold. In an exemplary embodiment, the antiplatelet agent is a member selected from hirudin, bivalirudin, lepirudin, desirudin, argatroban, dabigatran, dabigatran etexilate (oral formulation), melagatran, ximelagatran and prodrugs thereof. In an exemplary embodiment, the antiplatelet agent is a member selected from hirudin, hirudin analogs, bivalirudin, lepirudin and desirudin.

In an exemplary embodiment, the invention provides a stent comprising the following structure:

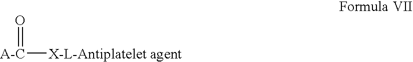

wherein A is a first fibrous polymer scaffold, X is a member selected from NH, L is polyethylene glycol, and the antiplatelet agent is hirudin, hirudin analog, bivalirudin, lepirudin and desirudin. In another exemplary embodiment, the C═O moiety is part of a lactide moiety in the first fibrous polymer scaffold. In another exemplary embodiment, the C═O moiety is part of a glycolide moiety in the first fibrous polymer scaffold.

In an exemplary embodiment, any of the stents described in this Summary or described herein comprises a second polymer which comprises a member selected from PTFE and Dacron. In an exemplary embodiment, any of the stents described in this Summary or described herein further comprising a sleeve which surrounds the exterior surface of the first fibrous polymer scaffold conduit or filled conduit, but is not located within the lumen of the first fibrous polymer scaffold conduit or filled conduit. In an exemplary embodiment, any of the stents described in this Summary or described herein said sleeve comprises a polymer or subunit which is a member selected from polyethylene terephthalate and polytetrafluoroethylene.

In an exemplary embodiment, for any of the stents described herein, a fibrous polymer scaffold is seamless. In another embodiment, the fibers of the scaffold are circumferentially aligned. In various embodiments, at least one of the fibers of the fibrous polymer scaffold comprises poly(lactide-co-glycolide) (PLGA). In certain embodiments, the fibers of the scaffold include an antiplately agent bound thereto, e.g., hirudin is covalently attached to the fibrous polymer scaffold by a linker which is a member selected from di-amino poly(ethylene glycol) and poly(ethylene glycol).

### Reversibly Associated Bioactive Materials

Generally, if it is desired that the biologically active agent remain active in the fiber for a long period of time, it is preferable to covalently attach the biologically active molecule to the fiber itself. In an exemplary embodiment, a bioactive agent is immobilized on a component (e.g., fibrin) of a fibrin sealant. In contrast, if it is desired that the biologically active agent escape the fiber (e.g., by diffusion from the fiber, erosion of the fiber, etc.), the agent should be reversibly associated with the fiber. The reversibly associated agent can, for example, be entrapped in a delivery matrix by adding the agent to the matrix components during manufacture of the matrix. In an exemplary embodiment, the agent is added to a polymer melt or a solution of the polymer. Other methods for reversibly incorporating agents into a delivery matrix will be apparent to those of skill in the art.

Examples of such reversible associations include, for example, agents that are mechanically entrapped within the matrix and agents that are encapsulated in structures (e.g., within microspheres, liposomes, etc.) that are themselves entrapped in, or immobilized on, the matrix. Other reversible associations include, but are not limited to, agents that are adventitiously adhered to the fiber by, for example, hydrophobic or ionic interactions and agents bound to one or more fiber component by means of a linker cleaved by one or more biologically relevant process. The reversibly associated agents can be exposed on the fiber surface or they can be covered with the same or a different fiber, such as a bioerodable polymer, as described below.

In an exemplary embodiment, the surface character of the fiber material is altered or manipulated by including certain additives or modifiers in the fiber material during its manufacture. A method of preparing surface-functionalized polymeric materials by this method is set forth in, for example, Caldwell, supra. In the Caldwell method, additives or modifiers are combined with the polymeric material during its manufacture. These additives or modifiers include compounds that have affinity sites, compounds that facilitate the controlled release of agents from the polymeric material into the surrounding environment, catalysts, compounds that promote adhesion between the bioactive materials and the fiber material and compounds that alter the surface chemistry of the fiber material.

As used herein, the term “affinity site” refers to a site on the polymer that interacts with a complementary site on a biologically active agent, or on the exterior surface of the structure to which the matrix is applied.

Affinity sites contemplated in the practice of the present invention include such functional groups as hydroxyl, carboxyl, carboxylic acid, amine groups, hydrophobic groups, inclusion moieties (e.g., cyclodextrin, complexing agents), biomolecules (e.g. antibodies, haptens, saccharides, peptides) and the like, that promote physical and/or chemical interaction with the bioactive agent or tissue. In the present embodiment, the affinity site interacts with a bioactive agent or tissue by non-covalent means. The particular compound employed as the modifier will depend on the chemical functionality of the biologically active agent and/or the groups on the surface of a particular tissue. Appropriate functional groups for a particular purpose can readily be deduced by one of skill in the art.

In another preferred embodiment, the fiber used in the invention is a substantially flowable material that can be delivered to a site of insult by means of, for example, a catheter, needle or other percutaneous delivery device. Preferred embodiments of the substantially flowable material are those that cure to a substantially non-flowable coating in vivo. Materials meeting these criteria include, for example, fibrin sealants, hydrophobic poly(hydroxy acids) and the like. The substantially flowable material will generally include one or more biologically active agents. The amount of a particular biologically active material contained in the substantially flowable material varies depending on a number of factors, including, for example, the activity of the agent and the tenaciousness with which the agent adheres to the delivery matrix.

In another preferred embodiment, the biologically active material interacts with a surfactant that adheres to the fiber material. Presently preferred surfactants are selected from benzalkonium halides and sterylalkonium halides. Other surfactants suitable for use in the present invention are known to those of skill in the art.

In a still further preferred embodiment, the bioactive material interacting with, and adhering to, the fiber material is a taxane, a taxane derivative or a combination thereof.

In another exemplary embodiment, a first molecule (which may or may not be a biomolecule) is covalently attached to the stent and/or polymer scaffold of the invention. This first molecule can be used to interact with a second biomolecule. In an exemplary embodiment, the first molecule is a linker, and the second biomolecule is a member selected from a receptor molecule, biochemical factor, growth factor and a differentiation factor. In an exemplary embodiment, the first molecule is a member selected from heparin, heparan sulfate, heparan sulfate proteoglycan, and combinations thereof. In an exemplary embodiment, the second biomolecule is a member selected from a receptor molecule, biochemical factor, growth factor and a differentiation factor. In another exemplary embodiment, the first molecule is covalently attached through a linker. In an exemplary embodiment, the linker includes a member selected from a water-soluble polymer and a water-insoluble polymer. In another exemplary embodiment, the linker includes a member selected from polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly(amino acids), e.g., poly(aspartic acid) and poly(glutamic acid); nucleic acids and copolymers thereof. In an exemplary embodiment, the linker includes a member selected from polyethylene glycol (PEG) and polypropylene glycol (PPG), or a mixture thereof. In another exemplary embodiment, the PEG or PPG comprises a number of monomeric subunits which is an integer from 1 to 5000. In an exemplary embodiment, said number of monomeric subunits is an integer from about 10 to about 1000. In an exemplary embodiment, said number of monomeric subunits is an integer from about 10 to about 500. In an exemplary embodiment, said number of monomeric subunits is an integer from about 20 to about 400. In an exemplary embodiment, said number of monomeric subunits is an integer from about 20 to about 250. In an exemplary embodiment, said number of monomeric subunits is an integer from about 50 to about 200. In an exemplary embodiment, said number of monomeric subunits is an integer from about 50 to about 125. In an exemplary embodiment, said number of monomeric subunits is an integer from about 50 to about 100. In an exemplary embodiment, said number of monomeric subunits is an integer from about 60 to about 90. In an exemplary embodiment, said number of monomeric subunits is an integer from about 60 to about 90. In an exemplary embodiment, said linker is a member selected from di-amino poly(ethylene glycol), poly(ethylene glycol) and combinations thereof. For biomolecules that do not bind to heparin, direct conjugation to the polymer scaffold or through a linker (such as PEG, amino-PEG and di-amino-PEG) is also feasible.

Biomolecules can be incorporated within the stents of the invention during electrospinning or post-fabrication. These biomolecules can be incorporated via blending, covalent attachment directly or through various linkers or by adsorption.

### Biodegradable and Bioresorbable Fiber Materials

Fiber compositions preferably have intrinsic and controllable biodegradability, so that they persist for about a week to about six months. The fibers are also preferably biocompatible, non-toxic, contain no significantly toxic monomers and degrade into non-toxic components. Moreover, preferred fibers are chemically compatible with the substances to be delivered, and tend not to denature the active substance. Still further preferred fibers are, or become, sufficiently porous to allow the incorporation of biologically active molecules and their subsequent liberation from the fiber by diffusion, erosion or a combination thereof. The fibers should also remain at the site of application by adherence or by geometric factors, such as by being formed in place or softened and subsequently molded or formed into fabrics, wraps, gauzes, particles (e.g., microparticles), and the like. Types of monomers, macromers, and polymers that can be used are described in more detail below.

Representative biodegradable polymers include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

Still further preferred fibers are water-insoluble materials that comprise within at least a portion of their structure, a bioresorbable molecule. An example of such a fiber is one that includes a water-insoluble copolymer, which has a bioresorbable region, a hydrophilic region and a plurality of crosslinkable functional groups per polymer chain.

For purposes of the present invention, “water-insoluble materials” includes copolymers that are substantially insoluble in water or water-containing environments. Thus, although certain regions or segments of the copolymer may be hydrophilic or even water-soluble, the copolymer molecule, as a whole, does not by any substantial measure dissolve in water or water-containing environments.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region is preferably hydrophobic. In another embodiment, however, the bioresorbable region may be designed to be hydrophilic so long as the copolymer composition as a whole is not rendered water-soluble. Thus, the bioresorbable region is designed based on the preference that the copolymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable fibers include, for example, synthetically produced resorbable block copolymers of poly(.alpha.-hydroxy-carboxylic acid)/poly(oxyalkylene, (see, Cohn et al., U.S. Pat. No. 4,826,945). These copolymers are not crosslinked and are water-soluble so that the body can excrete the degraded block copolymer compositions. See, Younes et al., J. Biomed. Mater. Res. 21: 1301 1316 (1987); and Cohn et al., J. Biomed. Mater. Res. 22: 993 1009 (1988).

Presently preferred bioresorbable polymers include one or more components selected from poly(esters), poly(hydroxy acids), poly(lactones), poly(amides), poly(ester-amides), poly(amino acids), poly(anhydrides), poly(orthoesters), poly(carbonates), poly(phosphazines), poly(phosphoesters), poly(thioesters), polysaccharides and mixtures thereof. More preferably still, the biosresorbable polymer includes a poly(hydroxy) acid component. Of the poly(hydroxy) acids, polylactic acid, polyglycolic acid, polycaproic acid, polybutyric acid, polyvaleric acid and copolymers and mixtures thereof are preferred.

In addition to forming fragments that are absorbed in vivo (“bioresorbed”), preferred polymeric fibers for use in the methods of the invention can also form an excretable and/or metabolizable fragment.

Higher order copolymers can also be used as fibers in the methods of the present invention. For example, Casey et al., U.S. Pat. No. 4,438,253, which issued on Mar. 20, 1984, discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other fibers based on lactic and/or glycolic acids can also be utilized. For example, Spinu, U.S. Pat. No. 5,202,413, which issued on Apr. 13, 1993, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a di-functional compound, such as, a diisocyanate, diacylchloride or dichlorosilane.

The monomers, polymers and copolymers of use in the present invention preferably form a stable aqueous emulsion, and more preferably a flowable liquid. The relative proportions or ratios of the bioresorbable and hydrophilic regions, respectively are preferably selected to render the block copolymer composition water-insoluble. Furthermore, these compositions are preferably sufficiently hydrophilic to form a hydrogel in aqueous environments when crosslinked.

The specific ratio of the two regions of the block copolymer composition for use as fibers in the present invention will vary depending upon the intended application and will be affected by the desired physical properties of the implantable fiber, the site of implantation, as well as other factors. For example, the composition of the present invention will preferably remain substantially water-insoluble when the ratio of the water-insoluble region to the hydrophilic region is from about 10:1 to about 1:1, on a percent by weight basis.

Preferred bioresorbable regions of fibers useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes.

As set forth above, the preferred composition also includes a hydrophilic region. Although the present composition contains a hydrophilic region, in preferred fibers, this region is designed and/or selected so that the composition as a whole, remains substantially water-insoluble.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene)oxide. Such poly(alkylene)oxides can include, for example, poly(ethylene)oxide, poly(propylene)oxide and mixtures and copolymers thereof.

Concerning the disposition of the biologically active agent in the fiber, substantially any combination of bioactive compound and fiber that is of use in achieving the object of the present invention is contemplated by this invention. In a preferred embodiment, the bioactive material is dispersed in a resorbable fiber that imparts controlled release properties to the biologically active agent. The controlled release properties can result from, for example, a resorbable polymer that is cross-linked with a degradable cross-linking agent. Alternatively, the controlled release properties can arise from a resorbable polymer that incorporates the biologically active material in a network of pores formed during the cross-linking process or gelling. In another embodiment, the drug is loaded into microspheres, which are themselves biodegradable and the microspheres are embedded in the fiber. Many other appropriate drug/fiber formats will be apparent to those of skill in the art.

In another preferred embodiment, an underlying polymeric component of a fiber of use in the invention is first impregnated with the biologically active material and a resorbable polymer is layered onto the underlying component. In this embodiment, the impregnated component serves as a reservoir for the bioactive material, which can diffuse out through pores in a resorbable polymer network, through voids in a polymer network created as a resorbable polymer degrades in vivo, or through a layer of a gel-like fiber. Other controlled release formats utilizing a polymeric substrate, a bioactive agent and a fiber will be apparent to those of skill in the art.

### Pharmaceutically Acceptable Excipients/Pharmaceutical Formulations

A pharmaceutically acceptable excipient can also be included in stents of the invention. In an exemplary embodiment, the invention provides a composition which includes a stent of the invention and a bioactive agent in conjunction with a pharmaceutically acceptable excipient or carrier. In an exemplary embodiment, the pharmaceutically acceptable excipient is a member selected from inert diluents, granulating and disintegrating agents, binding agents, lubricating agents, and a time delay material.

In another exemplary embodiment, the stents described herein are part of a kit. This kit can comprise an instruction manual that teaches a method of using the invention and/or describes the use of the components of the kit.

In certain exemplary embodiments, the invention provides a stent having the following characteristics. An exemplary vascular stent/stent graft, e.g., a peripheral vascular stent has a length of from about 5 to about 40 mm. In an exemplary embodiment, the peripheral vascular stent has a diameter from about 6 mm to about 15 mm. In an exemplary embodiment, the peripheral vascular stent includes nanofibers wound in a longitudinally aligned fashion. In various embodiments, the peripheral vascular stent includes one PLLA inner layer with s supporting PLLA and/or other polymer layers. In an exemplary embodiment, the fiber diameter is from about 1 micron to about 10 microns. In various embodiments, the fiber layer thickness is from a bout 1 to about 10 microns thick. Exemplary peripheral vascular stents have a stent scaffold with a thickness of from about 0.5 to about 1 mm microns thick. Exemplary stent scaffolds include those made of titanium, cobalt chromium, NiTiNOL, PLLA, PU or other polymer springs or rings. Various embodiments include from about 10% ge to about 15% ge biosorbable fiber, and from about 85% ge to about 90% ge nonsorbable fiber.

In an exemplary embodiment, the invention provides a carotid wall stent. In various embodiments, the carotid wall stent has a length of from about 10 to about 25 mm. In an exemplary embodiment, the diameter of the stent is from about 5 mm to about 10 mm. Exemplary stents include nanofibers wound in a longitudinally aligned fashion. Various exemplary stents include one PLLA inner layer with supporting PLLA and/or other polymer layers (e.g., PU, PVA). In various embodiments, the fiber diameter is from about 1 micron to about 10 microns. In various embodiments, the fiber layer thickness is from a bout 1 to about 10 microns thick. Exemplary peripheral vascular stents have a stent scaffold with a thickness of from about 0.5 to about 1 mm microns thick. Exemplary stent scaffolds include those made of titanium, cobalt chromium, NiTiNOL, PLLA, PU or other polymer springs or rings. Various embodiments include from about 10% ge to about 15% ge biosorbable fiber, and from about 85% ge to about 90% ge nonsorbable fiber.Fiber Orientation: Longitudinally aligned first layer wound inside

### The Methods

In various embodiments, the present invention provides methods of making the stents and stent scaffolds of the invention. In an exemplary embodiment, a stent scaffold is engaged with a mandrel of an electrospray device and a first layer of a nanodiamter polymer material is deposited onto the stent scaffold (FIG. 14). Exemplary stents produced by this method and those set forth below are shown in FIG. 12 and FIG. 13.

In an exemplary embodiment, the invention provides a method of forming a stent on a stent scaffold. The stent scaffold has an intraluminal and an extraluminal surface, a first longitudinal terminus and a second longitudinal terminus. The method includes, depositing a first layer of a plurality of nanodiameter polymer fibers on a mandrel (FIG. 17, FIG. 18). In an exemplary embodiment, the mandrel is attached to an electrospray device. The first layer of a plurality of nanodiameter polymer fibers is then contacted with an intraluminal surface of a stent scaffold such that the stent scaffold is disposed upon the mandrel and the first layer of a plurality of nanodiameter polymer fibers (FIG. 19). It is generally preferred that the intraluminal surface of the stent scaffold intimately contacts the first layer of a plurality of nanodiamter polymer fibers. In an exemplary embodiment, the contact between the first layer and the stent scaffold results in the first layer adhering to the intraluminal surface of the stent scaffold. In another exemplary embodiment, a second layer a plurality of nanodiameter polymer fibers is deposited on an extraluminal surface of the stent scaffold (FIG. 20). The second layer of a plurality of nanodiameter fibers is disposed upon and intimately contacts the extraluminal surface of the stent scaffold. It is generally preferred that the second layer of plurality of nanodiameter polymer fibers adheres to the extraluminal surface, the first layer and combinations thereof.

In another exemplary embodiment, the invention provides a method of forming a stent on a stent scaffold. The stent scaffold has an intraluminal and an extraluminal surface, and a first longitudinal terminus and a second longitudinal terminus. The method includes engaging the first longitudinal terminus of a stent scaffold with a rotating member. In an exemplary embodiment, the rotating member is a component of an electrospray unit that corresponds to a chuck fastening used to fix a mandrel to the electrospray device (e.g., a mandrel-anchoring chuck of an electrospraying device and said mandrel is not engaged with the mandrel-anchoring chuck) (FIG. 15). A first layer of a plurality of nanodiameter polymer fibers is deposited on the extraluminal surface of the stent scaffold. The first layer is disposed upon and intimately contacts the extraluminal surface of said stent scaffold. It is generally preferred that the first layer adheres to the extraliuminal surface of the stent scaffold (FIG. 16).

In another exemplary embodiment, the invention provides a method of forming a stent on a stent scaffold. The stent scaffold has an intraluminal and an extraluminal surface, and a first longitudinal terminus and a second longitudinal terminus. The method includes disposing within an intraluminal space of a stent scaffold defined by the intraluminal surface a nanodiameter polymer fiber deposition device (FIG. 21). Using the deposition device, a first layer of a plurality of nanodiameter polymer fibers is deposited onto the intraluminal surface, thereby forming a first intraluminal nanodiamter polymer fiber layer. An exemplary deposition device is a needle-like structure that is perforated along its longitudinal axis with multiple openings of a size sufficient to allow egress of the precursor of the layer of plurality of nanodiameter polymer fibers (FIG. 22). In various exemplary embodiments, the method further includes depositing on the extraluminal surface a second layer of a plurality of nanodiameter polymer fibers such that the resulting extraluminal layer of a plurality of nanodiameter fibers is disposed upon and intimately contacts the extraluminal surface of said stent scaffold.

In various embodiments according to each of the embodiments set forth above, at least one said layer of nanodiamter polymer fibers extends longitudinally beyond a member selected from said first longitudinal terminus, said second longitudinal terminus and a combination thereof.

In exemplary embodiments according to each of the methods above, the invention provides a method in which the stent scaffold or one or more layer of a plurality of nanodiameter polymer fibers includes one or more bioactive agent, e.g., hirudin. The bioactive agent can be covalently bound or otherwise non-covalently associated with a component of the stent of the invention.

In various exemplary embodiments, the nanofiber is deposited onto a flat metallic/polymer/material mesh and folded to a stent shape. The ends can be joined together, e.g., welded/glued/fastened to retain the stent shape. In an exemplary embodiment, nanofiber is deposited in an aligned or random form outside the stent to complete the structure.

In certain exemplary embodiments, the invention provides a method wherein at least one layer of nanodiameter polymer fibers covering partially the center or end parts of the stent. In an exemplary embodiment, the covering is longitudinal on a member selected from the first longitudinal terminus, the second longitudinal terminus and a combination thereof.

In various exemplary embodiments, the invention provides a method of supporting and/or repairing blood vessels. This method involves contacting a blood vessel in need of support and/or repair with a device of the invention.

In another aspect, the invention provides a method of treating a vascular disease such as atherosclerosis and stenosis. This method involves contacting the vascular disease site with a device of the invention. Exemplary vascular diseases treatable by this method include atherosclerosis, stenosis and a combination thereof.

In an exemplary embodiment, the invention provides a method of treating an injury comprising a severed anatomical structure of essentially tubular cross-section. An exemplary severed anatomical structure comprises a first severed stump and a second severed stump. The method includes interposing a stent of the invention between the first severed stump and the second severed stump such that both the first longitudinal terminus and the second longitudinal terminus contact a member selected from the first severed stump, a region of the anatomical structure distal to the first severed stump and combinations thereof and a member selected from the second severed stump, a region of said anatomical structure distal to the second severed stump and combinations thereof, respectively. On proper orientation of the device, the first longitudinal terminus is fastened to the member selected from the first severed stump, the region of the anatomical structure distal to the first severed stump and combinations thereof, and the second longitudinal terminus to the member selected from the second severed stump, a region of the anatomical structure distal to the second severed stump and combinations thereof, forming a patent anatomical structure, thereby treating the injury.

As will be appreciated by those of skill, the stents of the invention are deployable by an conventional method, including but not limited to catheters, partial or full vascular cutdowns and the like. In an exemplary embodiment, a stent of the invention is deployed by a guide wire or through a catheter to the appropriate/desired location in the body.

The invention is further illustrated by the Examples that follow. The Examples are not intended to define or limit the scope of the invention.

### EXAMPLES

### Example 1

### PLLA Nanofiber Scaffold Preparation

Biodegradable poly(L-lactide) (PLLA) (Lactel Absorbable Polymers, Pelham, Ala., 1.09 dL/g inherent viscosity) was used to fabricate nanofiber scaffolds by electrospinning. (Zong, X., Biomacromolecules, 4(2): 416-23 (2003)). Briefly, the PLLA solution (10% w/v in chloroform) was delivered by a programmable pump to the exit hole of the electrode at a flow rate of 25 μL/minute. A high-voltage supply (Glassman High Voltage Inc., High Bridge, N.J.) was used to apply the voltage at 20 kV. The collecting plate was on a rotating drum that was grounded and controlled by a stepping motor. To align the nanofibers, the electrospun scaffold was stretched uniaxially to 200% engineering strain at 60° C. Nanofibrous scaffolds were approximately 150 μm in thickness. The surface of the nanofibrous scaffold was coated with 2% gelatin or fibronectin (5 μg/cm²) before cell seeding. No significant difference in cell adhesion and morphology was detected between gelatin and fibronectin coating. Randomly-oriented scaffolds were used as controls.

Scanning electron microscopy (SEM) was used to visualize nanofiber alignment after uniaxial stretching. SEM images show that uniaxial stretching resulted in aligned nanofibers. The average nanofiber diameter in the scaffold was approximately 500 nm with an average gap size of approximately 4 μm.

### Example 2

### Longitudinally Aligned Polymer Scaffold Conduit

Biodegradable poly(lactic-co-glycolic-acid) (PLGA) (Lactel Absorbable Polymers, Pelham, Ala., 0.82 dL/g inherent viscosity) was used to fabricate nanofiber scaffolds by electrospinning. The PLGA solution (20% w/v in HFIP) was delivered by a programmable syringe pump to the exit hole of the electrode at a flow rate of 1 mL/hour. A high-voltage supply was used to apply the voltage at 11 kV. The collector substrate was a grounded steel mandrel attached to a motor capable of rotated the mandrel around its long axis. Teflon tape was wrapped around a section of the mandrel to create a non-conducting region. The mandrel was rotated at a slow speed (<15 rpm) as the PLGA fibers were electrospun. The jet alternated between the two sections of the mandrel separated by the non-conducting Teflon tape region resulting in deposition of PLGA fibers that were aligned parallel to the long axis of the mandrel. The rotation of the mandrel ensured even coverage of PLGA fibers around the mandrel. After electrospinning was completed, the edges of electrospun PLGA fibrous conduit were made blunt with a scalpel and the conduit was then removed from the Teflon tape and mandrel.

In order to verify fiber alignment, the tube was cut along its long axis and the fiber morphology was visualized with a light microscope. A majority of the fibers were aligned in the longitudinal direction (ie along the long axis of the conduit). It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A stent of essentially tubular cross-section bounded by a first longitudinal terminus and a second longitudinal terminus, said stent comprising:
a) a stent scaffold having an intraluminal surface and an extraluminal surface; and
b) a first nanodiameter fibrous polymer layer, comprising a plurality of nanodiameter polymer fibers, in intimate contact with a member selected from said intraluminal surface, said extraluminal surface and combinations thereof.

2. The stent according to claim 1, wherein said stent scaffold is formed of a material other than said nanodiameter polymer fibers.

3. The stent according to claim 1, further comprising a second nanodiameter fibrous polymer layer, comprising a plurality of nanodiameter polymer fibers, in intimate contact with said first nanodiameter fibrous polymer layer.

4. The stent according to claim 1, wherein said scaffold is disposed between said first layer and a second nanodiameter fibrous polymer layer, comprising a plurality of nanodiameter polymer fibers.

5. The stent according to claim 1 wherein each member of said plurality of nanodiameter polymer fibers of said first layer is aligned in an essentially parallel orientation with at least one other member of said plurality of nanodiameter polymer fibers.

6. The stent according to claim 3, wherein each essentially each member of said plurality of nanodiameter polymer fibers of said second layer is aligned in an essentially parallel orientation with at least one other member of said plurality of nanodiameter polymer fibers.

7. The stent according to claim 1 wherein said plurality of nanodiameter polymer fibers of said first layer is essentially randomly aligned.

8. The stent according to claim 3, wherein said plurality of nanodiameter polymer fibers of said second layer is essentially randomly aligned.

9. The stent of claim 1, wherein said fibers of said first nanodiameter fibrous polymer layer are aligned with an average angle of a member selected from between 0° and 1°, 0° and 5°, 0° and 10°, 0° and 15°, and 0° and 20°.

10. The stent of claim 3, wherein said fibers of said second nanodiameter fibrous polymer layer are aligned with an average angle of a member selected from between 0° and 1°, 0° and 5′, 0° and 10°, 0° and 15°, and 0° and 20°.

11. The stent of claim 1, wherein said first nanodiameter fibrous polymer layer is seamless.

12. The stent of claim 1, wherein said first nanodiameter fibrous polymer layer comprises a member selected from polydimethylsiloxane, polycaprolactone, polyalkylene oxide, polypeptide, polyurethane, polyvinyl alcohol, collagen, elastin, laminin, alginate, fibrin, aliphatic polyester, hyaluronic acid, proteoglycan, fibronectin and combinations thereof.

13. The stent of claim 13, wherein said aliphatic polyester is a member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid) and combinations thereof.

14. The stent of claim 12, wherein the polyalkylene oxide is a member selected from polyethylene oxide, polypropylene oxide and combinations thereof.

15. The stent of claim 12, wherein said first nanodiameter fibrous polymer layer comprises poly(lactide-co-glycolide) (PLGA) or poly L-lactide (PLLA).

16. The stent of claim 1, further comprising a cell interacting with a member selected from said stent scaffold and said first nanodiameter fibrous polymer layer.

17. The stent of claim 16, wherein said cell is embedded within said first nanodiameter fibrous polymer scaffold.

18. The stent of claim 16, wherein said cell is a member selected from an adult cell, a stem cell and a progenitor cell.

19. The stent of claim 18, wherein said cell is a member selected from an adult vascular cell, a vascular progenitor cell, and a vascular stem cell.

20. The stent of claim 1, further comprising a bioactive agent covalently attached to said first nanodiameter fibrous polymer layer.

21. The stent of claim 20, wherein said bioactive agent is covalently bound to said first nanodiameter fibrous polymer layer through a linker.

22. The stent of claim 21, wherein said bioactive agent is a member selected from an anticoagulant, an antiplatelet agent, a growth factor, a differentiation factor, and a combination thereof.

23. The stent of claim 21, wherein said linker comprises a member selected from a peptide, saccharide, poly(ether), poly(amine), poly(carboxylic acid), poly(alkylene glycol), poly(ethylene glycol), poly(propylene glycol), copolymers of ethylene glycol and propylene glycol, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), polysialic acid, polyglutamate, polyaspartate, polylysine, polyethyeleneimine, polylactide, polyglyceride and copolymers thereof, and polyacrylic acid.

24. A method of treating an injury comprising a severed anatomical structure of essentially tubular cross-section, wherein said severed anatomical structure comprises a first severed stump and a second severed stump, said method comprising:
(a) interposing a stent according to claim 1 between said first severed stump and said second severed stump such that both said first longitudinal terminus and said second longitudinal terminus contact a member selected from said first severed stump, a region of said anatomical structure distal to said first severed stump and combinations thereof and a member selected from said second severed stump, a region of said anatomical structure distal to said second severed stump and combinations thereof, respectively; and
(b) fastening said first longitudinal terminus to said member selected from said first severed stump, said region of said anatomical structure distal to said first severed stump and combinations thereof, and said second longitudinal terminus to said member selected from said second severed stump, a region of said anatomical structure distal to said second severed stump and combinations thereof, forming a patent anatomical structure, thereby treating said injury.

25. A method of treating a vascular disease, comprising contacting a site of said vascular disease with a stent of claim 1.

26. The method of claim 25, wherein said vascular disease is a member selected from atherosclerosis, stenosis and a combination thereof.

27. A method of forming a stent on a stent scaffold, said stent scaffold having an intraluminal and an extraluminal surface, a first longitudinal terminus and a second longitudinal terminus, said method comprising:
(a) depositing a first layer of a plurality of nanodiameter polymer fibers on a mandrel;
(b) contacting said first layer of nanodiameter polymer fibers with an intraluminal surface of a stent scaffold such that said stent scaffold is disposed upon and said intraluminal surface of said stent scaffold intimately contacts said first layer of nanodiamter polymer fibers; and
(c) depositing a second layer of a plurality of nanodiameter polymer fibers on an extraluminal surface of said stent scaffold such that said second layer of a plurality of nanodiameter fibers is disposed upon and intimately contacts said extraluminal surface of said stent scaffold.

28. A method of forming a stent on a stent scaffold, said stent scaffold having an intraluminal and an extraluminal surface, and a first longitudinal terminus and a second longitudinal terminus, said method comprising:
(a) engaging said first longitudinal terminus of a stent scaffold with a rotating member; and
(b) depositing a first layer of a plurality of nanodiameter polymer fibers on said extraluminal surface of said stent scaffold such that said first layer of a plurality of nanodiameter fibers is disposed upon and intimately contacts said extraluminal surface of said stent scaffold.

29. The method according to claim 28, wherein said rotating member is a mandrel-anchoring chuck of an electrospraying device and said mandrel is not engaged with said mandrel-anchoring chuck.

30. A method of forming a stent on a stent scaffold, said stent scaffold having an intraluminal and an extraluminal surface, and a first longitudinal terminus and a second longitudinal terminus, said method comprising:
(a) disposing within an intraluminal space of a stent scaffold defined by said intraluminal surface a nanodiameter polymer fiber deposition device; and
(b) depositing with said deposition device a first layer of a plurality of nanodiameter polymer fibers on a mandrel, thereby forming a first intraluminal nanodiamter polymer fiber layer.

31. The method of claim 30, further comprising:
(c) depositing on said extraluminal surface a second layer of a plurality of nanodiameter polymer fibers such that the resulting extraluminal layer of a plurality of nanodiameter fibers is disposed upon and intimately contacts said extraluminal surface of said stent scaffold.

32. The method according to claim 27, 28 or 30 wherein at least one said layer of nanodiamter polymer fibers extends longitudinally beyond a member selected from said first longitudinal terminus, said second longitudinal terminus and a combination thereof.
